(19) United States
(12) Patent Application Publication
(10) Pub. No.: US 2006/0264897 A1
(43) Pub. Date: Nov. 23, 2006

(54) Apparatus and method for delivering therapeutic and/or other agents to the inner ear and to other tissues

(75) Inventors: Thomas J. Lobl, Valencia, CA (US); Stephen J. McCormack, Claremont, CA (US); Thomas H.R. Lenarz, Hannover (DE); John V. Schloss, Valencia, CA (US); Anna Imola Nagy, Valencia, CA (US); Jacob E. Pananen, Los Angeles, CA (US)

Correspondence Address: BANNER & WITCOFF, 1001 G STREET N W, SUITE 1100, WASHINGTON, DC, 20001, US

(73) Assignee: NeuroSystec Corporation, Valencia, CA (US)

(21) Appl. No.: 11/337,815

(22) Filed: Jan. 24, 2006

Related U.S. Application Data
(60) Provisional application No. 60/665,368, filed on Mar. 28, 2005.
(60) Provisional application No. 60/645,755, filed on Jan. 24, 2005.
(60) Provisional application No. 60/645,756, filed on Jan. 24, 2005.
(60) Provisional application No. 60/645,757, filed on Jan. 24, 2005.
(60) Provisional application No. 60/645,606, filed on Jan. 24, 2005.

(51) Int. Cl. A61M 31/00; A61M 5/00; A61N 1/00; A61M 37/00

(52) U.S. Cl. 604/506; 604/117; 604/288.01; 607/137

(57) ABSTRACT

An apparatus may include a needle for sustained delivery of drugs and other agents to the inner ear or other tissues of a human or an animal. The needle can include an insertion stop, and can be placed through the round window membrane or through a surgically-prepared hole in a bone. The needle can be in fluid communication with a port and/or with a micro-infusion or osmotic pump. A cochlear implant electrode can be used instead of a needle.

### CROSS-REFERENCE TO RELATED APPLICATIONS

This application claims the benefit of U.S. Provisional Application Ser. Nos. 60/665,368, (filed Mar. 28, 2005 and titled “Apparatus and Method for Delivering Therapeutic Agents to the Inner Ear”), 60/645,755 (filed Jan. 24, 2005 and titled “Treatment of Inner Ear Disorders by Direct Cochlear Injection of NMDA Receptor Antagonists”), 60/645,757 (filed Jan. 24, 2005 and titled “Treatment of Inner Ear Disorders by Direct Cochlear Injection of Dextromethorphan”), 60/645,756 (filed Jan. 24, 2005 and titled “Treatment of Inner Ear Disorders by Direct Cochlear Injection of Subtype-Specific NMDA Receptor Antagonists”) and 60/645,606 (filed Jan. 24, 2005 and titled “Treatment of Inner Ear Disorders by Direct Cochlear Injection of Therapeutic Agents”). All of these applications are incorporated by reference herein.

### BACKGROUND OF THE INVENTION

It is well known that drugs work most efficiently in the human body if they are delivered locally at the place where the illness occurs. When delivered systemically there is a much greater chance for side effects as all tissues are exposed to large quantities of the drug. However, if the affected area is inside the body, localized drug delivery presents challenges. Either single doses or multiple doses can only be delivered to tissues located in anatomically difficult areas if a specialized injection device is used. This is especially true for injections into the cochlea, and other specific sub-cochlear locations in the inner ear.

Many therapeutics proposed for the treatment of tinnitus, neurological disorders that have tinnitus as a symptom, and other inner ear disorders have not been commercialized because of problems associated with systemic delivery. When administered orally or by intravenous injection, these agents are ineffective because they are rapidly metabolized, do not cross the blood-labyrinth barrier, and/or have undesirable side effects at other locations in the body that limit the dose employed. For example, corticosteroids, neurotrophins, anxiolytics, and ion channel ligands have substantial side effects.

Dextromethorphan ((+)-3-methoxy-N-methylmorphinan) is another example. Dextromethorphan has been proposed for the treatment of tinnitus (see U.S. Pat. No. 5,863,927). Because dextromethorphan is rapidly metabolized, however, co-administration of an inhibitor of its metabolism is thought to be necessary to achieve therapeutic levels. In addition, dextromethorphan can cause undesirable side effects when administered orally (e.g., blurred vision, confusion, fainting spells, insomnia, irregular heartbeat, palpitations, chest pain, irritability, nervousness, excitability, muscle or facial twitches, pain or difficulty passing urine, seizures, convulsions, severe nausea, vomiting, slurred speech, diarrhea, constipation, dizziness, drowsiness, hives, rashes, stomach upset, dry mouth, headache, and loss of appetite). The reason for such an extensive side-effect profile may be because of the non-selectivity of many NMDA antagonists for several other receptor types.

NMDA receptor antagonists are known to be effective in treating tinnitus and in preventing noise- or drug-induced hearing loss, and are generally neuroprotective by preventing apoptosis of neurons. Unfortunately, severe side effects are associated with higher doses of NMDA receptor antagonists (e.g., schizophrenia-like psychotic effects, motor ataxia and memory impairment) when they are administered orally or intravenously.

Therapeutic agents can be delivered to either the middle or inner ear tissues for the treatment of various diseases and conditions associated with inner ear tissue. Areas of the inner ear tissue structures where treatment can be beneficial include portions of the osseous labyrinth, such as the cochlea. However, the delivery of therapeutic agents to the inner ear in a controlled and effective manner is difficult due to the size and structure of the inner ear. The same is true of the anatomical structures which separate the middle ear from the inner ear (e.g. the round window membrane). The inner ear tissue is of such a size and location that it is only readily accessible through invasive microsurgical procedures.

Access to the osseous labyrinth in the inner ear, including the cochlea, is typically achieved through a variety of structures of the middle-inner ear interface including, but not limited to, the round window membrane. As is known, the middle ear region includes the air-containing zone between the tympanic membrane (the ear drum) and the inner ear. Currently, a variety of methods exist for delivering therapeutic agents to the middle and inner ear for the treatment of inner ear related diseases and conditions. These methods include drug injection through the tympanic membrane, surgically implanting drug loaded sponges and other drug releasing materials, and positioning drug delivering catheters and wicks within the middle ear. Although such conventional methods may ultimately result in the delivery of a therapeutic agent into the inner ear (e.g., by perfusion through the round window membrane), delivery of the therapeutic agent is generally not well controlled and the amount of the therapeutic agent that arrives within the inner ear is not known. Accordingly, there remains a need in the art for effective methods for sustained and controlled delivery of therapeutic agents to the inner ear.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In at least some embodiments, a device for delivering therapeutic (or other type) agents includes components such as a pump, filters and a fluid carrying system. Devices according to at least some embodiments can be used to deliver multiple bolus doses or continuous infusions of drugs (or other agents) to the human body over a longer period of time such as, but not limited to, a few days.

Various embodiments provide an apparatus and method for the controlled delivery of low volumes of therapeutic (or other type) agents into the cochlea. The apparatus and method can eliminate the need for extensive intrusive surgery. The agent(s) can be delivered and injected into the inner ear by an implanted apparatus. A fluid delivery system of the apparatus can include a catheter system that can extend through the ear canal, past the tympanic membrane, through the middle ear and into the cochlea through the round window. Alternatively, an agent can be delivered from an external pump through a subcutaneous port and catheter to a needle penetrating the temporal bone into the cochlea or through other bones to other regions (e.g., of the brain) avoiding the non-sterile middle ear region.

Apparatuses according to at least some embodiments will enable a physician to deliver therapeutic (or other type) agents into the inner ear for diseases best treated by a direct administration of the therapeutic agent(s) to this specific location. These apparatuses will also enable the physician to make one or multiple treatments over several days to the same location. The apparatuses described herein include a system that, when connected to a pump and syringe and then surgically placed by a physician, will enable convenient and sustained delivery of a variety of agents to the inner ear to treat hearing-related and other ailments such as tinnitus, infections of the inner ear, inflammatory diseases, inner ear cancer, acoustic neuroma, acoustic trauma, Ménière's Disease and the like.

### BRIEF DESCRIPTION OF THE DRAWINGS

The foregoing summary of the invention, as well as the following detailed description of preferred embodiments, is better understood when read in conjunction with the accompanying drawings, which are included by way of example, and not by way of limitation with regard to the claimed invention.

FIG. 1 is a schematic diagram of a first apparatus, according to at least some embodiments, for delivering agents to the inner ear.

FIG. 2 is a schematic diagram of a second apparatus, according to at least some embodiments, for delivering agents to the inner ear.

FIG. 3 is a schematic diagram of a third apparatus, according to at least some embodiments, for delivering agents to the inner ear.

FIG. 4 is a drawing of a fourth apparatus, according to at least some embodiments, for delivering agents to the inner ear.

FIG. 5 is a diagram of a syringe according to at least some embodiments.

FIG. 6 is a cross-sectional view of the barrel of the syringe in FIG. 5.

FIG. 7 shows female luer connector according to at least some embodiments.

FIG. 8 is a cross-sectional view of the connector in FIG. 7.

FIGS. 9-12 show quick disconnect fittings according to at least some embodiments.

FIGS. 13 and 14 show details of an inline micro-infusion filter used in at least some embodiments.

FIGS. 15-19 shows construction of an in-line filter assembly according to at least some embodiments.

FIGS. 20 and 21 show connectors for use in an in-line filter assembly according to additional embodiments.

FIGS. 22 and 23 show filter housings according to at least some embodiments.

FIGS. 24 and 25 show a suture anchor according to at least some embodiments.

FIG. 26 shows a round window injection needle according to at least some embodiments.

FIG. 27 shows a blunt injection needle according to at least some embodiments.

FIGS. 28-30 show injection needles according to additional embodiments.

FIG. 31 shows an injection needle according to another embodiment.

FIG. 32 is a cross-sectional view of the needle in FIG. 31.

FIG. 33 is a cross-sectional view of an injection needle according to another embodiment.

FIG. 34 is another cross-sectional view of the needle in FIG. 31.

FIG. 35 is a cross-sectional view showing a flanged end of a catheter assembly in an inlet or outlet of a micro/infusion filter.

FIG. 36 illustrates an example of a double lumen tubing for a catheter having two different inputs.

FIGS. 37 and 38 are cross-sectional views of catheters according to at least some additional embodiments.

FIGS. 39-45 illustrate subcutaneous ports according to at least some embodiments.

FIG. 46 shows a location for a subcutaneous port on a skull.

FIG. 47 shows a subcutaneous port and bone needle according to at least some embodiments.

FIG. 48 shows a bone needle according to at least some embodiments.

FIG. 49 shows a bone needle and osmotic pump according to at least some embodiments.

FIG. 50 is a schematic diagram of another apparatus, according to at least some embodiments, for delivering agents to the inner ear.

FIG. 51 is a partially schematic drawing of a cochlear implant electrode according to at least some embodiments.

FIG. 52 is a partial sectional view of the cochlear implant electrode of FIG. 51.

FIG. 53 is a schematic diagram of another apparatus, according to at least some embodiments, for delivering agents to the inner ear.

FIG. 54 is a schematic diagram of an additional apparatus, according to at least some embodiments, for delivering agents to the inner ear.

### DETAILED DESCRIPTION

A. Direct Injection of Therapeutics and Other Types of Agents to the Inner Ear.

At least some embodiments of the invention provide methods of treating inner ear disorders by using devices to inject therapeutic (and other type) agents directly into the cochlea. Direct injection into the cochlea overcomes a number of disadvantages of oral and other parenteral delivery methods. For example, drugs that have provided tinnitus relief and may do so by acting directly at the underlying molecular mechanisms responsible for tinnitus, include: clonazepam, alprazolam, memantine (see U.S. Pat. No. 6,066,652), cyclandelate, caroverine (see U.S. Pat. No. 5,563,140), lidocaine, tocainide and Neurontin (gabapentin). These drugs target various receptors responsible for neuronal signal transduction in the auditory system. Unfortunately, the side effects associated with the use of these drugs, at doses effective for tinnitus control, limit their use by oral or systemic administration. See Hester et al., 1998; Denk et al., 1997; Lenarz, 1986; Lenarz and Gulzow, 1985; Perucca and Jackson, 1985; Hulshof and Vermeij, 1985; Goldstein and Shulman, 2003.

Because the cochlea is beyond the blood brain barrier, however, a therapeutic agent directly placed at the cochlea will have access to hair cells, potentially the cerebrospinal fluid, the spiral ganglion, the auditory nerve and potentially other areas of the brain. Because the cochlea is a “closed” organ, lower doses of drug will be effective; this is both cost-effective and reduces the potential side effects of the drug. Thus, when the drug leaves the cochlea and enters the general circulation, the concentration of drug which may escape into the general circulation will be too small to cause either significant side effects or undesirable pharmacologic effects.

Inner ear disorders which can be treated by direct cochlear injection include but are not limited to tinnitus, noise-induced hearing loss, drug-induced hearing loss, chronic ear pain, Meniere's disease, neurodegeneration, physical (e.g., acoustic trauma or surgery) or chemical (e.g., aminoglycoside antibiotics) nerve damage, vertigo, TMJ, dental and facial nerve injury, hypersensitivity to chemicals and smells, and certain other neurological disorders relating to hypersensitivity diseases of nerves to stimuli for which tinnitus is a symptom. Direct injection of compounds into the cochlea makes possible development of compounds for drug therapy which would not otherwise be possible by other modes of delivery.

Therapeutic compounds which can be used to treat inner ear disorders according to the invention include those currently marketed as anxiolytics, anti-depressants, selective serotonin reuptake inhibitors (SSRI), calcium channel blockers, sodium channel blockers, anti-migraine agents (e.g., flunarizine), muscle relaxants, hypnotics, and anti-convulsants, including anti-epileptic agents. Examples of such compounds are provided below.

1. Anticonvulsants.

Anticonvulsants include barbiturates (e.g., mephobarbital and sodium pentobarbital); benzodiazepines, such as alprazolam (XANAX®), lorazepam, clonazepam, clorazepate dipotassium, and diazepam (VALIUM®); GABA analogs, such as tiagabine, gabapentin (an α2δ antagonist, NEURONTIN®), and β-hydroxypropionic acid; hydantoins, such as 5,5-diphenyl-2,4-imidazolidinedione (phenyloin, DILANTIN®) and fosphenyloin sodium; phenyltriazines, such as lamotrigine; succinimides, such as methsuximide and ethosuximide; 5H-dibenzazepine-5-carboxamide (carbamazepine); oxcarbazepine; divalproex sodium; felbamate, levetiracetam, primidone; zonisamide; topiramate; and sodium valproate.

2. NMDA Receptors as Therapeutic Targets for Tinnitus and Prevention of Nerve Cell Death.

The possible targets for direct tinnitus therapy, especially if drugs can be administered directly to the inner ear to avoid side effects, are voltage-gated Na⁺ channels, GABA_{A} receptor-linked chloride channels, other GABA receptors such as α2δ receptors, glutamate receptors (AMPA and NMDA receptors), and acetylcholine receptors (anticholinergics). The known effects of tinnitus drugs are distributed among these different types of receptors and ion channels. Although the primary target of lidocaine is voltage-gated Na⁺ channels, it also has some affinity for NMDA receptors. Caroverine blocks both AMPA and NMDA receptors, but has higher affinity for AMPA receptors, while memantine is selective for NMDA receptors. Blockage of AMPA receptors is more likely to interfere with hearing, while antagonists of NMDA receptors should also provide protection against excitotoxicity. Glutamate induced excitotoxicity results in the induction of apoptosis, with subsequent death of neurons and hair cells, that can result from excessive auditory stimulation of glutamatergic signaling. NMDA receptor antagonists prevent permanent hearing loss resulting from acoustic trauma or from ototoxic drugs, such as gentamycin or cisplatin. NMDA receptor antagonists would also be expected to prevent or reduce excitotoxicity associated with physical trauma, such as that associated with surgery. Memantine also blocks acetylcholine receptors. The anticholinergic effect of memantine has been proposed to be important to its inner ear pharmacology. Alprazolam enhances inhibitory GABAergic signals by increasing the affinity of GABA_{A} receptors for GABA. Gabapentin does not affect GABA_{A} receptors, but is thought to act as an agonist at GABA α2δ receptors. From a consideration of the pharmacology of drugs known to provide some benefit for tinnitus, NMDA receptors emerge as the most promising target. Although GABA_{A} and α2δ receptors may also be viable drug targets for inner ear therapy, the possibility remains that the benefit of these drugs would be indirect, acting by an anxiolytic mechanism, and not be suitable for direct delivery to the inner ear. The side effects associated with oral or systemic administration of any of these neuro-active drugs would preclude use of a dose that would ensure effective tinnitus control. See Sugimoto et al., 2003; Oestreicher et al., 1999; Oestreicher et al., 2002; Chen et al., 2004; Chen et al., 2003; Pujol and Puel, 1999; Kopke et al., 2002; Oestreicher et al., 1998; Nordang et al., 2000; Oliver et al., 2001; Galici et al., 1998; Costa, 1998; Stahl, 2004; Schwarz et al., 2005; Czuczwar and Patsalos, 2001; Taylor, 1997; Agerman et al., 1999; Basile et al., 1996; Duan et al., 2000; Guitton et al., 2004.

3. NMDA Receptor Antagonists.

There are many known inhibitors of NMDA receptors, which fall into five general classes. Each of the compounds described below includes within its scope active metabolites, analogs, derivatives, compounds made in a structure analog series (SAR), and geometric or optical isomers which have similar therapeutic actions.

4. Competitors for the NMDA Receptor's Glutamate Binding Site

Antagonists which compete for the NMDA receptor's glutamate-binding site include LY 274614 (decahydro-6-(phosphonomethyl)-3-isoquinolinecarboxylic acid), LY 235959 [(3S,4aR,6S,8aR)-decahydro-6-(phosphonomethyl)-3-isoquinolinecarboxylic acid], LY 233053 ((2R,4S)-rel-4-(1H-tetrazol-5-yl-methyl)-2-piperidine carboxylic acid), NPC 12626 (α-amino-2-(2-phosphonoethyl)-cyclohexanepropanoic acid), reduced and oxidized glutathione, carbamathione, AP-5 (5-phosphono-norvaline), CPP (4-(3-phosphonopropyl)-2-piperazine-carboxylic acid), CGS-19755 (seifotel, cis-4(phonomethyl)-2-piperidine-carboxylic acid), CGP-37849 ((3E)-2-amino-4-methyl-5-phosphono-3-pentenoic acid), CGP 39551 ((3E)-2-amino-4-methyl-5-phosphono-3-pentenoic acid, 1-ethyl ester), SDZ 220-581 [(αS)-α-amino-2′-chloro-5-(phosphonomethyl)-[1,1′-biphenyl]-3-propanoic acid], and S-nitrosoglutathione. See Gordon et al., 2001; Ginski and Witkin, 1994; Calabresi et al., 2003; Hermann et al., 2000; Kopke et al., 2002; Ikonomidou and Turski, 2002; Fisher et al., 2004; Danysz and Parsons, 1998.

5. Non-Competitive Inhibitors Which Act at the NMDA Receptor-Linked Ion Channel.

Antagonists which are noncompetitive or uncompetitive and act at the receptor-linked ion channel include amantadine, aptiganel (CERESTAT®, CNS 1102), caroverine, dextrorphan, dextromethorphan, fullerenes, gacyclidine (GK-11), ibogaine, ketamine, lidocaine, memantine, dizocilpine (MK-801), neramexane (MRZ 2/579, 1,3,3,5,5-pentamethyl-cyclohexanamine), NPS 1506 (delucemine, 3-fluoro-γ-(3-fluorophenyl)-N-methyl-benzenepropanamine hydrochloride), phencyclidine, tiletamine and remacemide. See Palmer, 2001; Hewitt, 2000; Parsons et al., 1995; Seidman and Van De Water, 2003; Danysz et al., 1994; Ikonomidou and Turski, 2002; Feldblum et al., 2000; Kohl and Dannhardt, 2001; Mueller et al., 1999; Sugimoto et al., 2003; Popik et al., 1994; Hesselink et al., 1999; Fisher et al., 2004.

6. Antagonists which Act at or Near the NMDA Receptor's Polyamine-Binding Site

Antagonists which are thought to act at or near the NMDA receptor's polyamine-binding site include acamprosate, arcaine, conantokin-G, eliprodil (SL 82-0715), haloperidol, ifenprodil, traxoprodil (CP-101,606), and Ro 25-6981 [(±)-(R,S)-α-(4-hydroxyphenyl)-β-methyl-4-(phenylmethyl)-1-piperidine propanol]. See Mayer et al., 2002; Kohl and Dannhardt, 2001; Ikonomidou and Turski, 2002; Lynch et al., 2001; Gallagher et al., 1996; Zhou et al., 1996; 1999; Lynch and Gallagher, 1996; Nankai et al., 1995; Fisher et al., 2004.

7. Antagonists which Act at the NMDA Recepotor's Glycine-Binding Site.

Antagonists which are thought to act at the receptor's glycine-binding site include aminocyclopropanecarboxylic acid (ACPC), 7-chlorokynurenic acid, D-cycloserine, gavestinel (GV-150526), GV-196771A (4,6-dichloro-3-[(E)-(2-oxo-1′-phenyl-3-pyrrolidinylidene)methyl]-1H-indole-2-carboxylic acid monosodium salt), licostinel (ACEA 1021), MRZ-2/576 (8-chloro-2,3-dihydropyridazino[4,5-b]quinoline-1,4-dione 5-oxide 2-hydroxy-N,N,N-trimethyl-ethanaminium salt), L-701,324 (7-chloro-4-hydroxy-3-(3-phenoxyphenyl)-2(1H)-quinolinone), HA-966 (3-amino-1-hydroxy-2-pyrrolidinone), and ZD-9379 (7-chloro-4-hydroxy-2-(4-methoxy-2-methylphenyl)-1,2,5,10-tetra-hydropyridanizo[4,5-b]quinoline-1,10-dione, sodium salt). Peterson et al., 2004; Danysz and Parsons, 2002; Ginski and Witkin, 1994; Petty et al., 2004; Fisher et al., 2004; Danysz and Parsons, 1998.

8. Antagonists which Act at the NMDA Receptor's Allosteric Redox Modulatory Site.

Antagonists which are thought to act at the allosteric redox modulatory site include oxidized and reduced glutathione, S-nitrosoglutathione, sodium nitroprusside, ebselen, and disulfiram (through the action of its metabolites DETC-MeSO and carbamathione). See Hermann et al., 2000; Ogita et al., 1998; Herin et al., 2001, Ningaraj et al., 2001; Kopke et al., 2002.

Some NMDA receptor antagonists, notably glutathione and its analogs (S-nitrosoglutathione and carbamathione), can interact with more than one site on the receptor.

CNQX (1,2,3,4-tetrahydro-7-nitro-2,3-dioxo-6-quinoxalinecarbonitrile) and DNQX (1,4-dihydro-6,7-dinitro-2,3-quinoxalinedione) bind to non-NMDA glutamate receptors. These and other antagonists or agonists for glutamate receptors can be used in the methods of the invention.

It is preferable that the NMDA receptor antagonists, like those disclosed herein, inhibit NMDA receptors without inhibiting AMPA receptors. The reason for this is that inhibition of AMPA receptors is thought to result in impairment of hearing. By contrast, selective inhibition of NMDA receptors is expected to prevent initiation of apoptosis, programmed cell death, of the neuron. Unlike AMPA receptors, which are activated by glutamate alone, NMDA receptors require a co-agonist in addition to glutamate. The physiologic co-agonist for NMDA receptors is glycine or D-serine. NMDA receptors but not AMPA receptors also bind reduced glutathione, oxidized glutathione, and S-nitrosoglutathione. Glutathione, γ-glutamyl-cysteinyl-glycine, is thought to bridge between the glutamate and glycine binding sites of NMDA receptors, binding concurrently at both sites. Activation of NMDA receptors leads to entry of calcium ions into the neuron through the linked ion channel and initiation of Ca²⁺-induced apoptosis. Intracellular calcium activates the NMDA receptor-associated neuronal form of nitric oxide synthase (nNOS), calpain, caspases and other systems linked to oxidative cell damage. Inhibition of NMDA receptors should prevent death of the neuron.

9. Subtype-Specific NMDA Receptor Antagonists.

A variety of subtype-specific NMDA receptor agonists are known and can be used in methods of the invention. For example, some NMDA receptor antagonists, such as arcaine, argiotoxin636, Co 101244 (PD 174494, Ro 63-1908, 1-[2-(4-hydroxyphenoxy)ethyl]-4-[(4-methylphenyl)methyl-4-piperidinol), despiramine, dextromethorphan, dextrorphan, eliprodil, haloperidol, ifenprodil, memantine, philanthotoxin343, Ro-25-6981 ([(±)-(R*,S*)-α-(4-hydroxyphenyl)-β-methyl-4-(phenylmethyl)-1-piperidine propanol]), traxoprodil (CP-101,606), Ro 04-5595 (1-[2-(4 chlorophenyl)ethyl]-1,2,3,4-tetrahydro-6-methoxy-2-methyl-7-isoquinolinol), CPP [4-(3-phosphonopropyl)-2-piperazinecarboxylic acid], conantokin G, spermine, and spermidine have moderate or high selectivity for the NR2B (NR1A/2B) subtype of the receptor. NVP-AAM077 [[[[(1S)-1-(4-bromophenyl)ethyl]amino](1,2,3,4-tetrahydro-2,3-dioxo-5-quinoxalinyl)methyl]-phosphonic acid] is an NR2A subtype-specific antagonist. See Nankai et al, 1995; Gallagher et al., 1996; Lynch and Gallagher, 1996; Lynch et al, 2001; Zhou et al., 1996; Zhou et al., 1999; Kohl and Dannhardt, 2001, Danysz and Parsons, 2002.

10. Useful Therapeutics Other than NMDA Receptor Antagonists.

Other useful therapeutic agents include nortriptyline, amytriptyline, fluoxetine (PROZAC®), paroxetine HCl (PAXIL®), trimipramine, oxcarbazepine (TRILEPTAL®), eperisone, misoprostol (a prostaglandin E1 analog), and steroids (e.g., pregnenolone, triamcinolone, methylprednisolone, and other anti-inflammatory steroids).

Each of these compounds includes within its scope active metabolites, analogs, derivatives, compounds made in a structure analog series (SAR), and geometric or optical isomers which have similar therapeutic actions.

11. Identifying Other Therapeutic Agents.

Two main approaches can be used to identify other compounds of therapeutic interest: non-behavioral responses (indirect quantitative measures) and behavioral responses. Non-behavioral responses can be assessed for example by measuring the neural response to a sound in the presence and absence of a test compound and following the treatment of an experimental animal or a tissue with salicylate to induce an increase in spontaneous neuronal firing. Examples of such measurements include, but are not limited to, measurements of compound action potential (CAP) and distortion product auto-acoustic emission (DPOAE).

Behavioral responses include conditioned responses to sound which correlate with behavior following high doses of salicylate. For example, an animal's response is compared before and after administering a test compound.

Tinnitus can be assessed in animal models before and after administration of a test compound. Methods for measuring tinnitus in animal models are described in Moody, “Animal Models of Tinnitus,” in Snow, Jr., ed., Tinnitus: Theory and Management, Chapter 7, pp 80-95, BC Decker, London, 2004 and in the references cited in the chapter.

12. Pharmaceutically Acceptable Formulations and Doses.

Therapeutic agents typically are injected in a pharmaceutically acceptable formulation. Pharmaceutically acceptable formulations typically are free of pyrogenic substances and are sterile to minimize adverse reactions. They may include other components such as buffers, artificial perilymph, saline or Ringer's solution.

Typical doses of a therapeutic agent will depend on the therapeutic agent itself as well as on the nature and severity of the inner ear disorder to be treated. Doses include, but are not limited to, 1 micromolar (μM) to 3.3 millimolar (mM) solution (e.g., 100-200 μM could be used with gacyclidine), with volumes delivered of these concentrations from 10 nL/hr to 200 microL/hour depending on the therapeutic drug or other agent used and its potency. Typical doses of dextromethorphan or a dextromethorphan-related compound range from 1-200 μM, with 50 μM a preferred dose. Either single or multiple injections or continuous infusions can be made. Determining whether a single injection or multiple injections or continuous infusions are necessary in a particular patient or experimental animal is well within the skill of the ordinary physician.

If desired, two or more therapeutic agents can be injected. These can be in the same formulation or in different formulations. Different agents can be injected at the same time or sequentially. For example, the therapeutic drug (or other agent) selected from above can be mixed from separate formulations together or co-formulated together in a separate vial with an antibiotic or an anti-inflammatory agent such as a steroid (dexamethasone, triamcinolone, etc.) and injected into the cochlea using devices according to at least some embodiments to achieve a desired therapeutic effect on tinnitus and an inflammatory condition.

B. Examples of Apparatuses and Methods for Direct Agent Delivery.

In at least some embodiments, direct cochlear injection of the above-described (and other) agents is accomplished with a device that is largely external to the patient. A needle is inserted through the ear canal or through the temporal bone. A catheter attached to the needle extends to the outside of the patient. The remainder of the device is also outside the patient, and thus under the control of a physician or the patient. In other embodiments, the injection device is connected through the skin to a subcutaneous port located on the mastoid bone or other convenient location and remains implanted completely inside the patient. Drugs (or other agents) are delivered through the subcutaneous port into the catheter assembly with a needle and ultimately into the cochlea.

In some embodiments, the injection device includes (1) a pumping system which can be adjusted to deliver between 1 nanoliter/hour through 200 microliters/hour and which can be turned off and on as needed to meet the needs of the patient; (2) a system which can be programmed to different flow rates depending on the therapeutic need of the patient; (3) a reservoir or syringe system which will hold the therapeutic drug or other agent and is connected to the pump in such a way that when the pump is running, the intended agent will be delivered to the patient; (4) a tubing assembly which is connected to the reservoir/syringe system and contains as needed sterile filters with a pore size sufficiently small to exclude bacterial and other common infectious organisms; and (5) a needle assembly. Optionally, quick disconnects and fittings to connect the tubing to the reservoir and the needle assembly can be included. In certain embodiments, a needle in the needle assembly is between 20 and 35 gauge (e.g., 28-31 gauge), is straight or bent between 90 and 180 degrees (e.g., 120 degrees), has a blunt tip or a bevel tip of between 0 and 75 degrees (e.g., 50 to 70 degrees), and may optionally have an insertion stop welded or otherwise attached to the needle to prevent over insertion of the needle into the cochlea. In some embodiments, the insertion stop is between 0.5 and 4 mm (e.g., between 1 and 3 mm) from the tip. Further details of apparatuses according to some embodiments are provided below. However, the described embodiments are merely examples. The invention includes embodiments in addition to those specifically described herein.

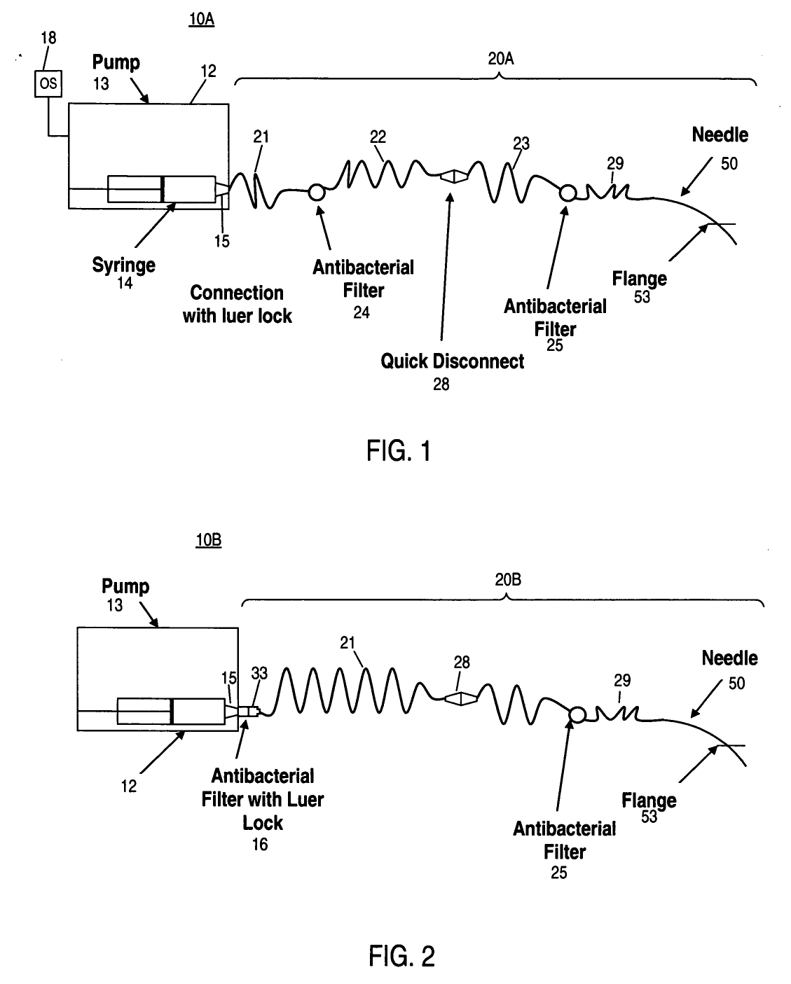

FIG. 1 is a schematic diagram of an apparatus 10A for delivering therapeutic (and other type) agents to the inner ear. Apparatus 10A includes an supply system 12 having an external micro-pump 13 with a syringe 14. Syringe 14 includes a male luer tip 15 that can act as a reservoir and hold, e.g., a therapeutic agent for treating ear ailments. A fluid carrying system 20A is attached to supply system 12. Fluid carrying system 20A includes catheter sections 21-23, in-line antibacterial filters 24 and 25 that provide sterility to the system and any agent(s) introduced through the system, an in-line quick-disconnect coupling 28, and a catheter section 29. Fluid carrying system 20A is in fluid connection with, and downstream of, supply system 12. As used herein (including in the claims), “downstream” refers to a direction from a source (such as syringe 14) to an outlet of a needle. Fluid carrying system 20A includes a proximal end connected to supply system 12 and a distal end carrying a needle 50 for introducing agent(s) into the inner ear of the patient. Various components of apparatus 10A are described in more detail below. A plunger of syringe 14 is connected to a screw mechanism (not shown) within pump 13 that drives the plunger to expel a drug (or other agent) from the syringe. Operating system 18 interacts with pump 13 to instruct the pump how to deliver the drug or other agent.

FIG. 2 is a schematic diagram of an apparatus 10B which is also for delivering agents to the inner ear. Like components of apparatus 10A and apparatus 10B have common reference numbers. Unlike apparatus 10A, fluid carrying system 20B of apparatus 10B includes a connector 16, which can be of the type manufactured by Filtertek, that includes an inline antibacterial filter. Connector 16 has an upstream end with a female luer tip that is attached to male luer tip 15 of syringe 14. The female luer tip of connector 16 has a standard size that enables easy connection to male tip 15, with the resulting interface between connector 16 and syringe 14 forming a connection which can be readily broken and remade. In at least one embodiment, the filter within connector 16 is a 0.22 micron membrane filter.

The filter in connector 16 (which can be a micro-infusion filter) is positioned downstream of, and is in fluid communication with, supply system 12. Any material exiting supply system 12 passes through the filter, allowing the filter to retain bacteria that might have penetrated into the sterile syringe 14, thereby preventing such bacteria from entering other parts of apparatus 10B or the patient. A downstream end of connector 16 is in fluid communication with catheter 21, placing catheter 21 in fluid communication with supply system 12. In some embodiments (and as shown in FIG. 2), connector 16 is coupled to catheter 21 via another connector 33, with connector 33 being attached to catheter 21. Connector 33 is described in more detail below. In other embodiments, there is no additional connection fitting between filter-containing fitting 16 and catheter 21. For example, catheter 21 can also be permanently secured (e.g., with adhesive) to connector 16. Various components in FIG. 2 are described in more detail below.

The configuration shown in FIG. 2 enables a physician to aseptically and rapidly fill syringe 14 using an attached needle from a sterile vial containing a formulated drug (or other agent) in solution or reconstituted lyophilized drug (or other agent) in solution, remove the syringe needle aseptically and attach sterile fluid carrying system 20B via connector 16 and its inline filter. This enables the physician to be confident that the drug or other agent being delivered into apparatus 10B would be sterile at least until the point of the quick disconnect.

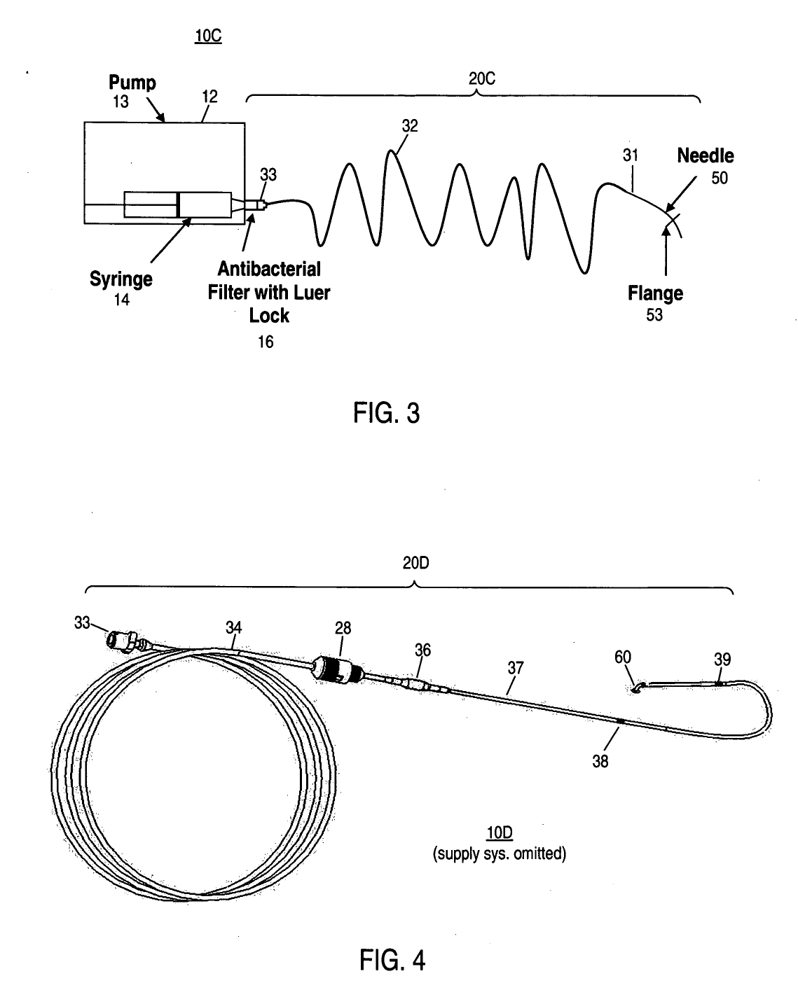

FIG. 3 is a schematic diagram of another apparatus 10C for delivering agents to the inner ear. Fluid carrying system 20C does not include an in-line quick disconnect coupling or an in-line filter. Although apparatus 10C only contains one sterilizing filter (i.e., the filter within connector 16), more than one filter could be used. An in-line filter could be positioned at any point along fluid line 32. FIG. 3 shows catheter 32 connected to a fitting 33, with that fitting 33 connected to fitting 16. In variations on the embodiment of FIG. 3, connector 16 is connected directly to catheter 32 (i.e., there is no intermediate connector coupling catheter 32 to connector 16). In still other variations, connector 33 is connected directly to male luer connection 15 on syringe 14 (i.e., connector 16 is omitted).

FIG. 4 is a drawing of another apparatus 10D for delivering agents to the inner ear. For simplicity, only the fluid carrying system 20D of apparatus 10D is shown. Fluid carrying system 20D is connectable to, e.g., supply system 12. Apparatus 10D includes a female luer connector 33 for connection (directly or via other components) to a male luer 15 in syringe 14 (not shown) within pump 13 (also not shown), catheter 34 (connected to female luer connector 33), in-line quick-disconnect coupling system 28 (connected to catheter 34), antibacterial filter assembly 36 (connected to quick-disconnect coupling system 28), catheter 37 (connected to antibacterial filter assembly 36), and injection needle assembly 60 (connected to catheter 37). Catheter 37 further includes suture anchors 38 and 39. Additional details of apparatus 10D are provided below.

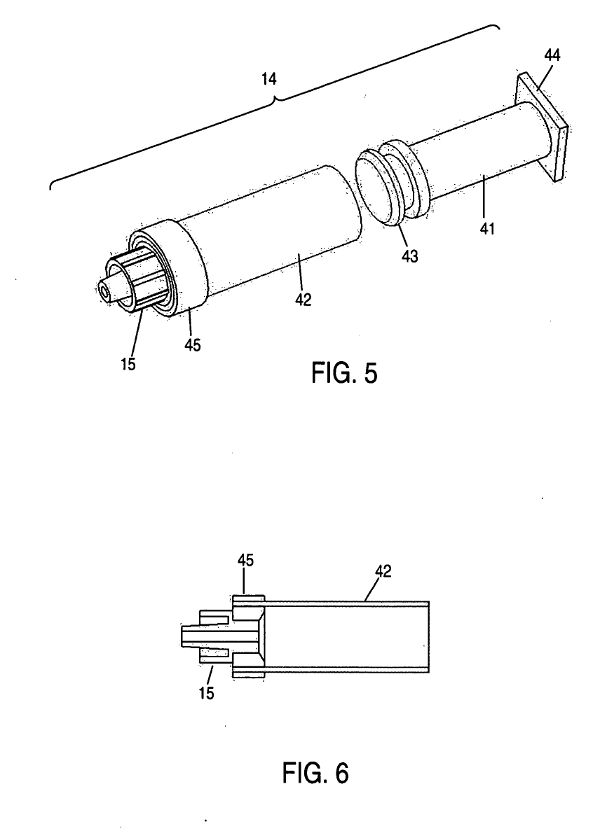

Pump 13 of apparatuses 10A-10D can deliver a variety of compatible liquid-formulated therapeutic (or other type) agents. Pump 13 includes a screw mechanism (not shown) that operates on syringe 14 by pushing a syringe plunger 41 into a syringe barrel 42 (described below in conjunction with FIGS. 5 and 6). Other known manners of incrementally advancing a plunger could also be used. A manual or computer controlled operating system 18 (not shown in FIG. 2 or 3) for pump 13 determines and controls when and how far plunger 41 will move within syringe barrel 42. The settings for the operation of plunger 41 are time and volume based. Operating system 18 of pump 13 controls volume by time and a motor turning the screw mechanism that pushes syringe plunger 41 within barrel 42. The volume delivered, therefore, correlates with the number of turns or portion of a turn per unit of time. In at least one embodiment, pump 13 can be set to deliver as little as 1 microliter/step (when the step defines how much the screw mechanism turns/unit time). For example, pump 13 and syringe 14 could deliver therapeutic agent(s) at a rate of 0.05 to 1 μL/step with a minimum of one step per hour or more depending on settings and variables and syringe diameter. Alternatively the pump could be used to deliver bolus injections or intermittent infusions of variable lengths of time and frequency.

For delivery to other neurological tissues, the volume can be set to higher volume delivery rates as is needed to provide the desired effects. For long term infusions it may be optimal to have even smaller delivery rates such as in the range of 10-100 nanoliters/hour delivery rates and conceivably even less. For gacyclidine, only about 10-100 nL/hr need be delivered to inhibit tinnitus if the contained drug or other agent was at the correct concentration.

One example of a commercially available pump that could be used for pump 13 is the Medtronic MiniMed Series 508 pump available from Medtronic MiniMed of Northridge, Calif. Other conventional pumps that operate in the same manner, but provide different therapeutic delivery rates, can also be used. The operating system of this pump would be reconfigured to provide the injection criteria discussed above. These conventional pumps can also be altered to provide flexible timings, delivery options and screw mechanisms to allow a different step size (and thereby change the volume delivered per step).

FIG. 5 shows syringe 14 in more detail. Syringe 14 includes a plunger 41 and a tubular barrel portion 42. FIG. 6 is a cross-sectional view of barrel 42 taken along its longitudinal centerline. Plunger 41 includes a stopper 43 disposed at one end to prevent fluid leakage past the inner wall of barrel 42. The stopper 43 end of plunger 41 is inserted into barrel 42. Stopper 43 may include one or more rings made of an elastomeric material and which engage an inner surface of barrel 42 to create a liquid tight seal, thus allowing fluid ejection when force is applied to the end 44 of plunger 41.

Syringe 14 is designed for positioning within a syringe compartment (or chamber) of pump 13. A drive member (e.g., a screw mechanism as previously described) within that chamber engages end 44 of plunger 41 and displaces plunger 41 to administer medication (or other agent) to the patient. Syringe 14 is designed to meet the operational specifications of the pump within which it will be installed. In particular, syringe 14 is sized and shaped in accordance with the requirements of the pump to be used, and friction forces attributable to sliding plunger seals, etc. are maintained within acceptable tolerances. Determining the proper size, shape and other characteristics of a syringe for use with a designated type of pump is within the routine ability of a person skilled in the art (once such person is provided with the information herein). In the embodiments shown, syringe 14 includes a male luer tip 15 for mating with a female luer tip in a connector attached to fluid carrying system 20A, 20B, 20C or 20D. Male luer tip 15 can be a locking or non-locking compression fitting.

Syringe barrel 42 can be manufactured from lightweight molded plastics suitable for disposal after a single use. Barrel 42 may have a fluoropolymer or any other biocompatible/drug compatible polymer inner layer or coating to provide drug compatibility. Stopper 43 is also formed from a fluoropolymer. As used herein (including the claims), “fluoropolymer” includes (but is not limited to) drug-compatible polymers selected from (but not restricted to) the group of fluoropolymers that include: PTFE (polytetrafluoroethylene, e.g., Algoflon®, Daikin-Polyflon®, Teflon®, Hostaflon®, Fluon®), ECTFE (ethylene-chlorotrifluoroethylene copolymer, e.g., Halar®), ETFE (ethylene-tetrafluoroethylene copolymer, e.g., Aflon®, Halon ET®, Hyflon®, Neoflon®, Tefzel®), FEP (tetrafluoroethylene-hexafluoropropylene copolymer, e.g., Neoflon®, Teflon®), MFA (tetrafluoroethylene perfluoro(methylvinyl ether) copolymer, e.g., Hyflon®), PCTFE (polychloro tri-fluoro ethylene, e.g., Aclon®, Neoflon®, Kel F®), PFA (perfluoroalkoxyethylene, e.g., Aflon®, Hyflon®, Neoflon®, Teflon®, Hostaflon®), and PVDF (polyvinylidene fluoride, e.g., Hylar®, Neoflon®, Kynar®, Foraflon®, Solef®). In order to reduce the sliding friction forces of plunger 41 inside barrel 42, the inner surface of barrel 42 may be prelubricated and the syringe stopper (and/or o-rings, if present) may be lubricated with a chemically inert fluoropolymer lubricant. Fluoropolymer lubricant reduces frictional forces while maintaining drug compatibility within syringe 14. Reduction of friction forces within syringe 14 is desirable for syringes used in a programmable medication infusion pump having a battery operated (and relatively low power) drive. Syringe 14 can be used without lubricant, but in such case the frictional forces are increased.

Barrel 42 can be molded as a single unit combined with male luer fitting 15. Alternatively, barrel 42 and fitting 15 can be manufactured as two or more separate components and glued together to make a tight connection. In cases where the barrel and male luer fitting are not glued together (e.g., if glue would not be drug compatible), a metal band 45 can be placed on the outside of the barrel to clamp the end of the barrel around the fitting and form a liquid-tight seal between the barrel and the luer component.

In other embodiments barrel 42 can be entirely manufactured from a fluoropolymer or another polymer which will provide superior biocompatibility and drug compatibility. The inner surface of such a barrel may also be lubricated with a fluoropolymer lubricant to reduce the sliding frictional forces between the syringe barrel and stopper. Plunger 41 and stopper 43 can also be manufactured from a fluoropolymer. In still other embodiments barrel 42 can be manufactured from glass, with the inner wall of the glass barrel acid-washed to improve drug compatibility. The plunger of a glass-barreled syringe can be made from glass, metal, or any drug-compatible polymer. As used herein (including the claims), “metal” includes metal alloys. The stopper for such a plunger could be manufactured from glass with a drug compatible o-ring fitting to make a leak tight seal, a fluoropolymer, or any other biocompatible/drug compatible polymer.

End 44 of plunger 41 is designed to fit within the pump syringe chamber and to mate with the pump drive assembly that pushes plunger 41 into barrel 42. In the example of FIG. 5 a square end (compatible with a MiniMed insulin pump) is shown. Other pumps can be used, although a different configuration of plunger end may be required.

In addition to mating with a female luer connector, male luer connector 15 fits within a holder assembly (not shown) of pump 13. Certain pumps may require an extended neck on the male luer connector in order for the syringe to mate properly with (and be held by) the syringe pump chamber. As with other syringe features, selection and/or design of a proper male luer connector for compatibility with a particular pump is within the routine ability of a person skilled in the art (once such person is provided with the information herein).

In some embodiments, a syringe and catheter are permanently connected. In such embodiments, a tube hole is formed in a closed end of the barrel, and the catheter is inserted into that hole and glued to form a permanent connection. Such an arrangement adds additional sterility protection, but may be harder to fill and prime.

Although the outer dimensions of syringe 14 may require standardization (so as to mate with a selected pump), the internal dimensions can be varied so as to vary the amount of agent dispensed from the syringe. For example, the diameter of stopper 43 and barrel 42 can be adjusted to control the amount of therapeutic agent(s) delivered during each operating step. In one embodiment, syringe 14 has a volume of approximately 90 nL/step. Syringes having a volume of greater than 90 nL/step can also be used. For example, a syringe according to another embodiment could deliver approximately 111 nL/step/hr. One embodiment of a syringe delivering approximately 111 nL/step/hr has a stopper diameter of 4 mm and a barrel having an ID of 4 mm, an outside diameter (OD) of 14 mm and a length of 37 mm. The locking neck on that embodiment has a length of 5 mm and an OD of 6 mm.

Syringes with smaller delivery rates are also contemplated. In certain embodiments, syringe 14 has delivery volumes significantly smaller than 90 nL/step, and can be modified to include splitters or other pumping methods such as osmotic or MEMS (microelectromechanical systems) pumps (e.g. piezo electric pumps with check valves, mini-peristaltic and other kinds of miniature pumps) containing the appropriate microfluidics. The advantages of a MEMS pump include the ability to turn it off and on as needed and the flexibility of varying the amount of liquid-formulated therapeutic delivered. An advantage of an osmotic pump is the ability to deliver very small volumes but in a continuous stream. However, osmotic pumps are not easily turned off unless they are designed with a closable door to the semi-permeable membrane.

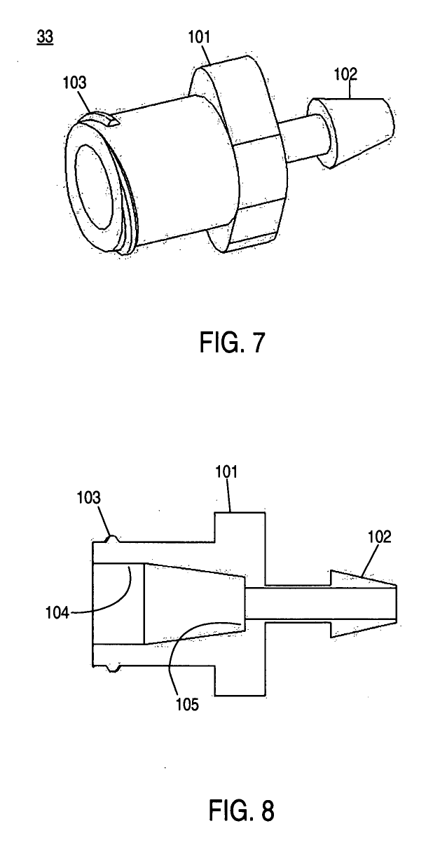

FIG. 7 shows female luer connector 33. Connector 33 is made of a fluoropolymer. In other embodiments the material for connector 33 can be selected from a group comprising biocompatible/drug compatible polymers such as other nylon, polypropylene, polysulfone, polyester, or other polymers. FIG. 8 is a cross-sectional view of connector 33 taken along its longitudinal centerline. Middle portion 101 is designed to allow the connector to be handled and twisted easily. Connector 33 includes a barb 102 at its downstream end for connection to catheter 21, 32 or 34. As described in more detail below, the external portion of a catheter is in some embodiments formed from silicone. Silicone expands when exposed to certain solvents, allowing easy insertion of barb 102 into an end of the catheter. When the solvent evaporates, the silicone returns to a smaller diameter and closes around barb 102 to make a tight seal. In some embodiments, epoxy, or other biocompatible adhesives can be used to strengthen and seal the connection between barb 102 and a catheter. In still other embodiments, connector 33 lacks a barb. Instead, the downstream end of the connector may include a flanged tip, a straight tube or a hole into which connective catheter tubing can be inserted and glued, molded or otherwise attached. For these and other embodiments, the female luer connector may be attached to a catheter using adhesive bonding, solvent bonding, clamping, flanging, ultrasonic welding, or the like.

The upstream end of female luer connector includes threads 103 for connection with male luer 15 of syringe 14. Other embodiments (not shown) may include a simple flange that is compatible with a corresponding type of male luer lock assembly.

As indicated above in connection with apparatus 10B (FIG. 2), a filter may be positioned within female luer connector 16 so that any material exiting syringe 14 will pass through the filter before entering catheter 21. In some embodiments, the internal structure of filter-containing luer connector 16 is similar to that of (non-filter-containing) connector 33 of FIGS. 7 and 8. In particular, female luer connector 16 has a slightly elongated internal cavity (similar to cavity 104 of connector 33, as shown in FIG. 8), with the filter secured at an end wall (similar to wall 105 shown in FIG. 8).

Apparatuses 10A, 10B and 10D of FIGS. 1, 2 and 4, respectively, include an in-line quick disconnect fitting 28 similar to that described in U.S. Pat. No. 5,545,152. Quick disconnect fitting 28, which can be used at any connection point along the fluid delivery portion of the apparatus, allows a physician or attendant to quickly and easily separate the supply system 12 from the remainder of the apparatus. For example, a physician can first insert needle 50 or 60 and an internal portion of a catheter attached to that needle into a patient's ear, and then subsequently attach supply system 12 and the remainder of the fluid carrying system using quick disconnect 28. Quick disconnect 28 also provides a quick and efficient manner for temporarily removing the “heavy” pump and cumbersome external portions of the apparatus when the needle and catheter remain within the patient's ear for an extended period of time (e.g., during sleeping, showering, etc.).

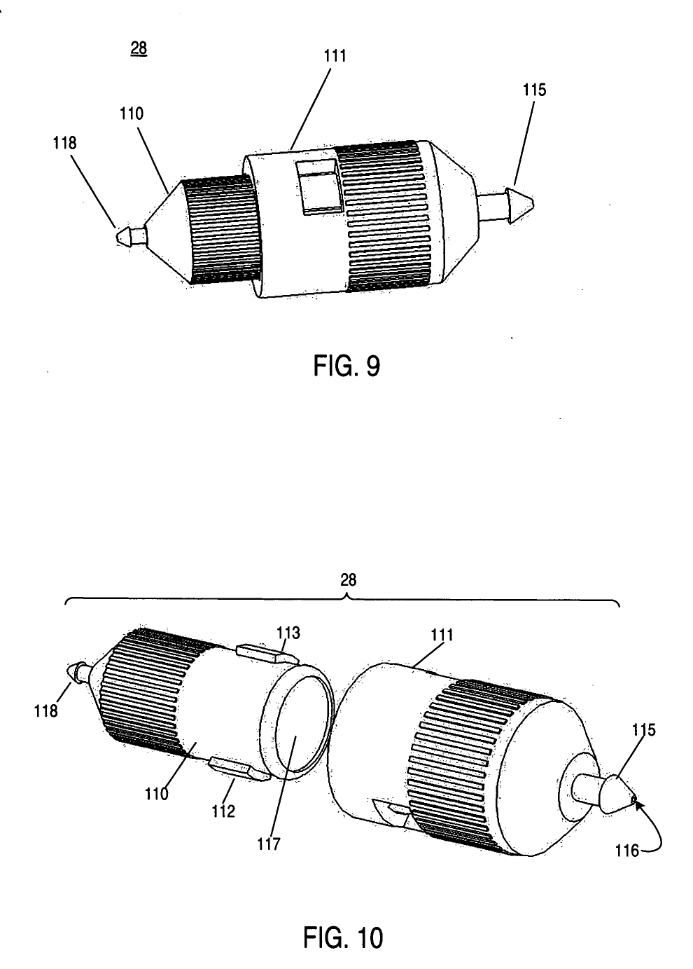

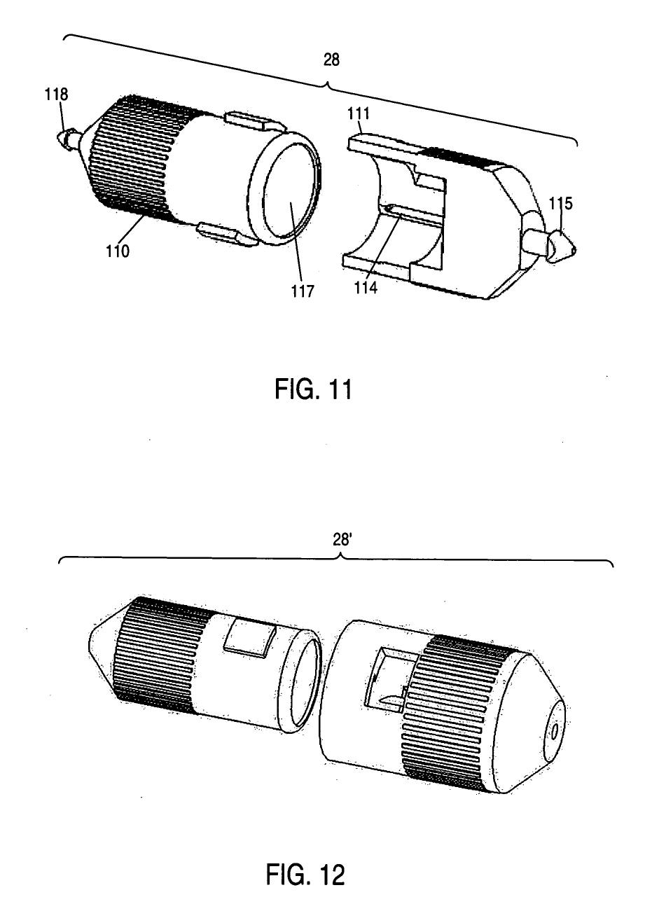

FIGS. 9-11 show in-line quick disconnect fitting 28 in more detail. FIG. 9 shows the male component 110 and female component 111 when joined. FIG. 10 shows components 110 and 111 separated. FIG. 11 is similar to FIG. 10, but with a portion of female component 111 removed to show internal features.

In some embodiments female component 111 is on the upstream side of the fluid carrying system (e.g., mounted to catheter 21, 22 or 34). In other embodiments, however, male component 110 is on the upstream side. Female component 111 has a generally cylindrical, open ended shape with a connector needle 114 mounted therein. Needle 114 is in fluid communication with a channel 116 inside of barb 115, which is in turn connected to a catheter (not shown in FIGS. 9-12). Channel 116 and other internal fluid passageways of female connector 111 in communication with needle 114 are lined with a fluoropolymer or other biocompatible/drug compatible polymer material. Connector needle 114 is recessed within female connector 111 to prevent accidental contact therewith, thereby avoiding accidental needle sticks and damage to needle 114, and increasing sterility protection. When female connector 111 and male connector 110 are joined, needle 114 penetrates septum 117 on male component 110. Septum 117 is formed from, e.g., a silicone elastomer. A needle and septum arrangement allows maintenance of a sterility barrier on the needle injection assembly side of the device while exchanging the syringe and contents therein.

Male component 110 includes a generally tubular nose adapted for side-fit connection within the receiving cavity of female component 111. Radial tabs 112 and 113 on male component 110 slide freely into radially open ports (not shown) formed in female component 111. The longitudinal slide-fit connection of the male and female components occurs in a response to relatively minimal longitudinal force. When the components are fully engaged in the longitudinal direction, the male component can be rotated within the female component toward a locked position. When coupling disconnection is desired, the male component can be back-rotated within the female component; the male and female component can be separated easily with a minimal longitudinal force. Quick disconnect coupling 28 provides a safe and easy disconnection and subsequent reconnection of an infusion fluid source, such as pump 13. The fluid contacting inner surface of the male component can also be lined with a fluoropolymer or an alternative biocompatible/drug compatible polymer. The needle within the female component is sufficiently long that when the male and female components are connected the needle penetrates the septum sufficiently to allow free fluid communication with the remainder of the device.

In at least one embodiment, quick disconnect coupling 28 includes barbs 115 and 118 or flanges (not shown) to assist in providing a strong link between the quick disconnect components and the upstream and downstream catheters. In other embodiments, the quick disconnect components may have holes for a catheter to be inserted. FIG. 12 shows one such embodiment (quick disconnect fitting 28′). In other embodiments, the connect/disconnect mechanism may incorporate a spring-loaded tab or latch which allows a slide-fit connection without any rotation necessary for locking. In such a mechanism, when the male component is inserted into the female component, a latch in the female component engages a groove or slot on the male component, locking the assembly together and at the same time allowing 360° swiveling. The two components can then be separated easily by pressing a tab, sliding a socket, or the like. In another embodiment the male may have o-rings to help make a tight seal and connection with the female component obviating the need for a septum or needle. Still other embodiments for connecting two kinds of tubing together with a sleeve that holds the two parts together and maintains sterility in a leak proof environment could also be used.

Although quick disconnect coupling 28 may be made from a fluoropolymer to provide superior drug compatibility, it may also be made of PVC, urethane and other thermoplastic elastomers, polyethylenes, nylons, acetals, polycarbonates, and various other polymers. In some embodiments, the male component includes a self sealing septum having a cut, cross cut or hole in the middle. The septum could also be removable. In another embodiment, the male component of the quick disconnect could also have a 3-D antibacterial filter imbedded within the housing; so that all fluid will pass through the filter into the catheter, obviating the need for a separate in-line 3-D filter assembly elsewhere.

The use of an inline quick disconnect 28 provides a physician with the ability to separate a positioned round window needle from a pump for the convenience of the patient. At the time the physician or attendant wants to reconnect the supply system 12 to the patient, a sterile needle of one component will be attached to a sterile septum (which septum may be wiped with a sterilizing solution such as alcohol) on the other component. Upon reconnection the sterile formulated drug (or other agent) solution can again flow to the cochlear-implanted needle from the pump and its syringe.

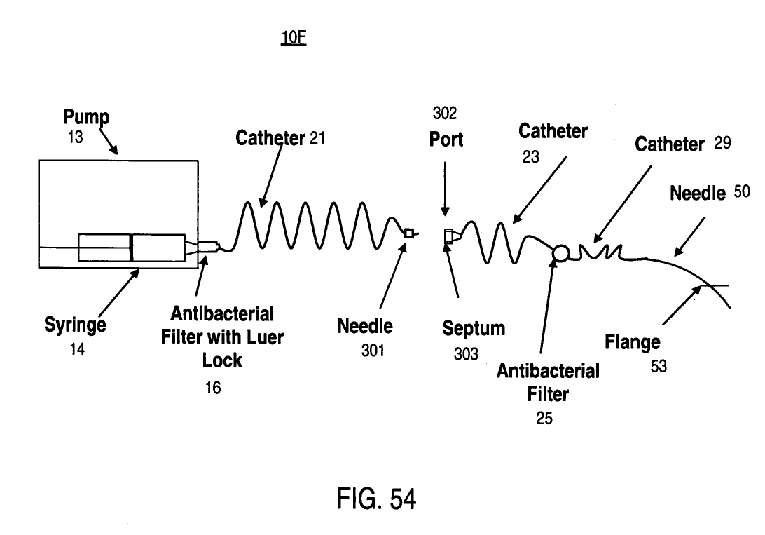

Other types of quick disconnect fittings may be used. For example, a coupling having a septum-piercing needle may not be recessed (e.g., the needle may not lie within a cavity such as in female component 111 of FIGS. 9-12). Rather than using a quick disconnect fitting to provide a sterile connection, a sterile needle of one component can be used to pierce the sterile septum of a port (see FIG. 54), where the port has a similar function to subcutaneous ports described later but is on a catheter outside the patient.

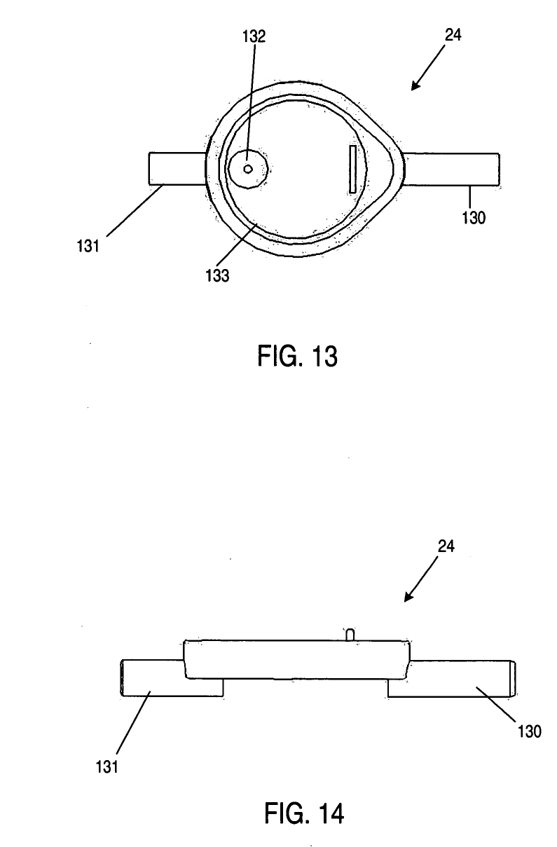

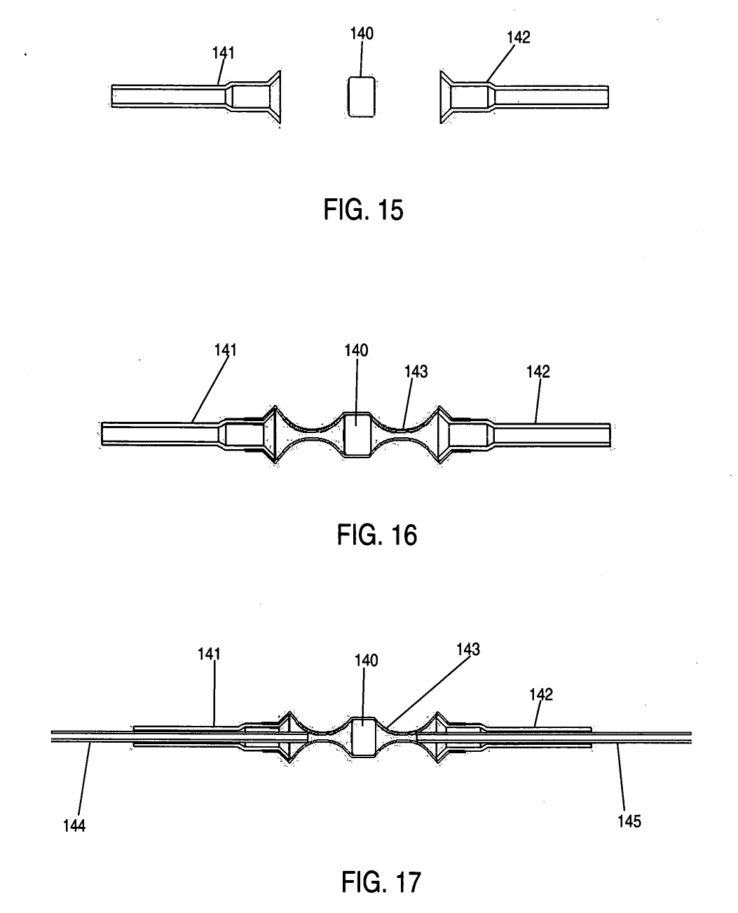

As shown in FIGS. 1, 2 and 4, various types of in-line filters may be employed. FIGS. 13 and 14 show additional details of inline micro-infusion filters 24 and 25. Only filter 24 is shown in FIGS. 13 and 14, with filter 25 being substantially the same or of an alternative design (such as is shown in FIG. 15-19). Filter 24 (available from Pall Corporation under the trade name Micro IV), provides either a primary or secondary antibacterial filter to ensure that a formulated drug or other material(s) being delivered to a patient through an implanted catheter and needle will be free of bacteria. Filter 24 includes an upstream connector (i.e., an inlet) 130 and a downstream connector (i.e., an outlet) 131 so that a fluid line (e.g., catheters 21 and 22) can be in fluid communication with and through the filter. Filter 24 includes a degassing hole 132 and an enclosed membrane filter element 133 with a filter pore size of 0.22 microns. This size will remove most bacteria to improve the safety of the filter.

A membrane filter may best be used where the filter remains external to the patient. However, membrane filters may clog easily. If implanted, a clogged membrane filter may be difficult to replace. Moreover, a membrane filter lacks dimensional strength and must be held in a housing with tube connections for attachment to a catheter. Membrane filters are usually limited to short-term use. Alternative embodiments of antibacterial filters include those that do not have membranes. For longer term use, a 3-D filter assembly may be substituted for a membrane filter. In particular, a three dimensional (3-D) filter element is a practical and robust filter with the dimensional strength useful for a variety of medical devices (including surgically applied injection devices and implanted biomedical applications) wherever an antibacterial filter is needed. Because of its dimensional strength, a 3-D filter element can be used “naked” (i.e., without additional housing) in a catheter or contained within a housing.

A 3-D filter element may be formed in various manners. In some embodiments, a 3-D filter element is formed by cutting or punching a filter element from a sheet of material (e.g., a biocompatible polymeric material or porous metallic material) with an appropriately small pore/channel size (such as <2 micron) for use as an anti-bacterial filter, and with the sheet having a thickness that will yield a filter element of a length that can extend along a flow path for several millimeters. The pore size can be <10 microns, e.g., <2.0 microns or <0.22 microns. A metallic 3-D filter element can also be formed by sintering, as described below. A 3-D filter element (however formed) can then be incorporated into a fluid system in any of a variety of ways. For example, a 3-D filter element can be inserted into a portion of a catheter or other tube (e.g., a catheter formed in part from a flexible biocompatible polymer such as silicone rubber) that is swollen (with a solvent) to allow easy insertion of the filter element into that tube. When the solvent evaporates, the tubing returns to its design diameter and closes around the filter element to make a tight seal. This tight seal prevents bacteria from getting around the filter element and forces the fluid to pass through the filter element interior. The outside of the 3-D filter element can also be glued or sealed with the tubing to prevent leakage around the sides of the filter element. Other techniques for forming a filter from a 3-D filter element can also be employed; some such techniques are discussed below.

Anti-bacterial filter assembly 36, positioned downstream of quick-disconnect 3 (see FIG. 4), is shown in a cross-sectional view in FIG. 19. Filter 36 includes a metallic 3-D disc filter element 140. As with the above-described filters, filter element 140 removes cells in a passing fluid to render the efflux sterile. This is important to the safety of the patient on which an infusion set is being used. One example of many ways a metal 3-D filter element can be prepared is as follows. A fine metal powder such as titanium metal (with the particle diameter selected for the desired resulting pore size) is tightly packed into a mold with the desired shape for the final filter element. The metal is heated to the point at which the powder particles begin to melt and form attachments to neighboring particles. This results in an intricate porous bonded meshwork which works like a filter, has a tortuous path and has a predetermined macro-external shape. A filter element can alternately be formed from type 316 stainless steel or any other biocompatible metal. As indicated above, metal (as used herein, including the claims) includes metal alloys. As indicated above, a 3-D filter element can alternatively be formed from a porous polymeric material having a pore size appropriate for an anti-bacterial filter. Without limitation and as further examples, a 3-D filter element (whether metallic or polymeric) can have a diameter in the range of about 0.010 inches to 0.400 inches (e.g., about 0.062 inches). The length of a 3-D filter element can be approximately 0.010 inches to 0.200 inches (e.g., about 0.039 inches). The pore size can be, e.g., <10 microns, <2.0 microns or <0.22 microns. Filter elements of other dimensions are acceptable (depending on the application and the device desired) as long as they function as an antibacterial filter; effective pore size is generally more critical than the overall dimensions. Smaller pore sizes increase back pressure.

FIGS. 15-19 are cross-sectional views (taken along the longitudinal centerlines of the components of filter 36) showing one method of constructing filter 36. FIG. 15 shows 3-D filter element 140 and two flared metal connectors 141 and 142. Filter element 140 and metal connectors 141 and 142 will be wrapped in tubing to form filter assembly 36. Metal connectors 141 and 142 are made from 316 stainless steel in some embodiments, but may also be formed from titanium, other types of stainless steel, and other metals. The outer diameters of the flared ends range from about 0.030 inches to 0.300 inches (e.g., around 0.080 inches). The outer diameters of the opposite ends of the metal connector range from about 0.020 inches to 0.200 inches (e.g., around 0.030 inches). The length of each connector ranges from about 0.1 inches to 1.0 inches (e.g., around 0.25 inches).

In FIG. 16, heat-shrink tubing 143 has been placed over metal connectors 141 and 142 and filter element 140. Heat is then applied so as to fully encase connectors 141 and 142 and filter element 140 in tubing 143. In some embodiments, heat-shrink tubing 143 is made of PTFE; other possible drug compatible materials include FEP, PFA and other fluoropolymers, polyester, polyolefin or other polymers. The expanded inner diameter of heat-shrink tubing 143 should be larger than the diameters of filter element 140 and the flared end diameters of connectors 141 and 142. The length of heat-shrink tubing 143 varies from about 0.25 inches to 2.0 inches (e.g., around 0.5 inches).

In FIG. 17, catheter tubing 144 and 145 is inserted into the non-flared ends of metal connectors 141 and 142. Catheter tubing 144 and 145, which connects the filter assembly to the rest of apparatus 10D, is formed from PTFE, FEP, PFA, other fluoropolymers, silicone, polyimide, PVC, polyurethane and/or other biocompatible and drug compatible polymers. Catheter tubing 144 and 145 may be bonded to metal connectors 141 and 142 using an epoxy or adhesive elastomer. Tubing 144 and 145 may be of different sizes, with the inside diameters of connectors 141 and 142 each corresponding to the inserted tubing.

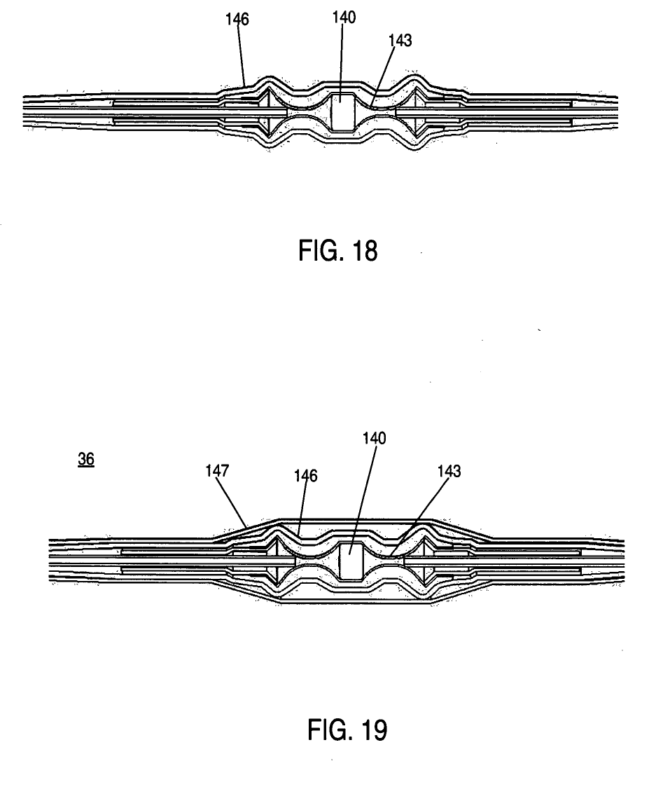

In FIG. 18, a larger tube 146 fully encases heat-shrink tubing 143, filter element 140, metal connectors 141 and 142, and the ends of catheter tubing 144 and 145. Tube 146 is formed from a flexible polymer, such as silicone rubber, which expands when exposed to certain solvents and then contracts when the solvent(s) evaporates. In at least some embodiments, the inner diameter of tube 146 varies from about 0.010 inches to 0.100 inches (e.g., around 0.020 inches).

In FIG. 19, an additional tube 147 has encased the remainder of the filter assembly. In some embodiments, tube 147 is made of a flexible biocompatible polymer (e.g., silicone rubber) and has an inner diameter between about 0.020 inches to 0.200 inches (e.g., 0.030 inches).

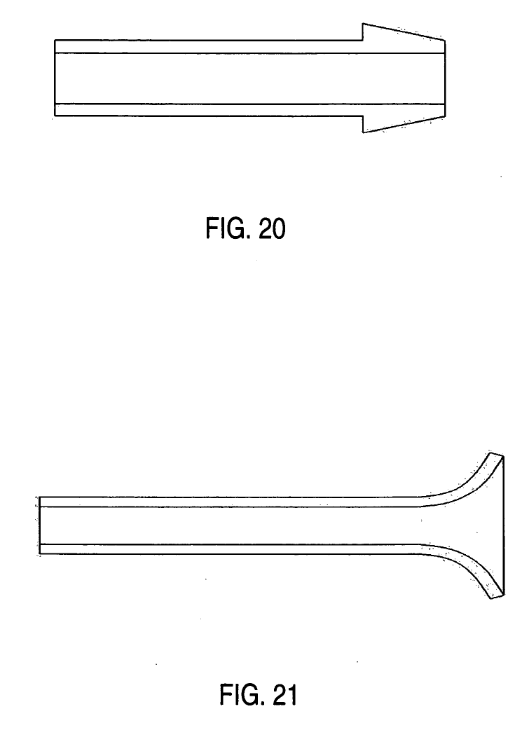

FIGS. 20 and 21 show metal connectors which are used instead of connectors 141 and 142 in other embodiments. For convenience, only one connector is shown in each of FIGS. 20 and 21, with the other connector of a pair being substantially identical (although perhaps of different dimensions). The connector pieces in FIGS. 20 and 21 are designed to provide a tight connection with the filter when they are encased in heat-shrink tubing. The embodiment FIG. 20 includes barb-shaped tubes, with the barbs facing the filter element when assembled. The embodiment of FIG. 21 includes flared tubes that face the filter element when assembled. Other embodiments (not shown) include a tube with a flange, where the flange is welded to the tubing shaft using known methods in the art such as laser welding. Alternatively, the flange (which may be plastic or metal) can be attached with epoxy, or other kinds of glue or adhesives. Additionally, if the internal hole of the flange is sized correctly, it can be heated to enlarge the hole and a tube sized correctly for the hole can be inserted to the correct depth, with the flange then allowed to cool and make a tight seal around the tube.

The connectors of FIGS. 20 and 21 (as well as connectors 141 and 142 of FIGS. 15-19) can be made of hard plastic, stainless steel, titanium, or other metals (e.g., 316 stainless steel). The connectors of FIGS. 15-19 could alternatively be formed from biocompatible and drug compatible polymers/plastics such as fluoropolymer, urethane, and other thermoplastic elastomers, polyethylenes, nylons, acetals, polycarbonates, and various other polymers.

In at least some other embodiments, an in-line filter includes a 3-D filter element within a housing that surrounds the filter element. One advantage of a housing is simplified removal and replacement of a filter. This may be especially valuable for implanted filters that should be operational for long periods of time inside an animal or person. A housing also serves to provide a tight seal around the filter element in order to prevent bacteria from getting around the filter element sides, thus forcing the fluid to pass through the filter element interior. A housing can include an upstream connector (inlet) and a downstream connector (outlet) so that the fluid line can be in fluid communication with and through the filter.

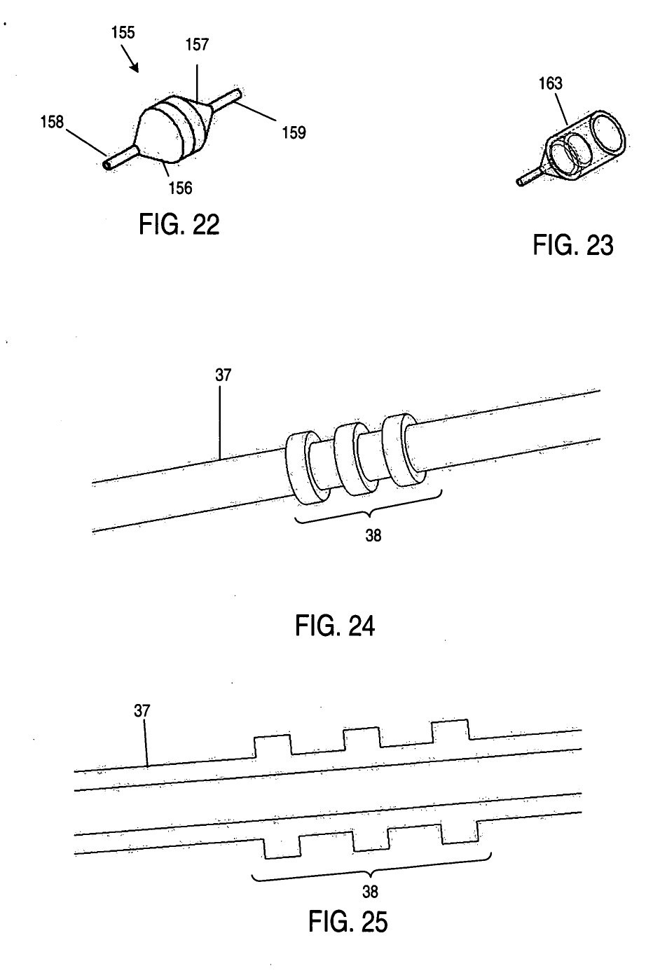

One embodiment for a three-dimensional filter housing 155 is illustrated in FIG. 22. Housing 155 consists of two metallic flared tubes 156 and 157 that are welded to a filter element and to each other. The weld is intended to provide a tight seal around the filter element. Alternatively, a filter element may be bonded to metal tubes 156 and 157 using a biocompatible, drug compatible epoxy or adhesive elastomer. Housing 155 can be made of any biocompatible metal such as 316 stainless steel or titanium. Housing 155 could also be made of biocompatible and drug compatible polymers/plastics such as fluoropolymer, urethane, and other thermoplastic elastomers, polyethylenes, nylons, acetals, polycarbonates, and various other plastics. The upstream inlet connector 158 and the downstream outlet connector 159 may have different diameters depending on the geometries of the catheters on either side. In one embodiment, the outer diameter of the inlet and outlet connectors varies from about 0.010 inches to 0.200 inches (e.g., about 0.012 inches), with the inner diameter of the flared ends of tubes 156 and 157 depending on the size of the filter element (e.g., between 0.010 inches and 0.200 inches).

Another embodiment for a three-dimensional filter housing consists of a single flared metal tube 163, as shown in FIG. 23. Preferably, a filter element is welded to the inside of tube 163, but the filter element may alternatively be bonded to tube 163 using an epoxy or adhesive elastomer. In another embodiment the filter element may be sintered from metal powder directly inside tube 163 rather than transferring an already-formed filter element into tube 163. Tube 163 can be made of any biocompatible metal such as 316 stainless steel or titanium. In other embodiments, tube 163 may be made of biocompatible polymers/plastics such as fluoropolymer, urethane, and other thermoplastic elastomers, polyethylenes, nylons, acetals, polycarbonates, and various other plastics. In use, the small end of tube 163 is attached to one catheter, and the flared end attached to another (larger) catheter. Dimensions of the tube ends will vary depending on the diameter of the connecting catheters and of the filter element.

An alternative embodiment of the three-dimensional filter element housing consists of a straight metallic tube (not shown). A filter element may be welded to the inside of the straight tube housing, may be bonded to the housing using an epoxy or adhesive elastomer, or may be sintered directly from metal powder directly into the housing. The housing can be made of any biocompatible metal such as 316 stainless steel or titanium, or from biocompatible, drug compatible polymers/plastics such as fluoropolymer, urethane, and other thermoplastic elastomers, polyethylenes, nylons, acetals, polycarbonates, and various other polymers. The inner diameter of the housing depends on the size of the filter element (e.g., between 0.010 inches and 0.200 inches).

In another embodiment, and as described in more detail below, a filter may be built into a subcutaneous port to provide sterility of the fluid that is introduced into the implanted port. In still other embodiments, a molded filter is designed to have a specific shape for a given location, e.g., a cup filter (for an injection port or a subcutaneous port) or a cylindrical filter (for a tube or other location). A filter can be removable for cleaning or replacement or it can be permanently attached to the device in which it is placed.

Referring again to FIG. 4, catheter 37 includes anchoring elements 38 and 39 designed to prevent lateral movement of catheter 37 once it has been secured with suture thread. Sutures may be used to attach catheter 37 to tissue in the middle ear so as to prevent the injection needle from slipping out of the round window membrane.

FIG. 24 is a perspective view illustrating suture anchor 38, with suture anchor 39 being substantially the same. FIG. 25 is a cross-sectional view of suture anchor 38 taken along the longitudinal centerline of catheter 37. Suture anchors 38 and 39 are molded directly to catheter 37 using a liquid silicone elastomer or another suitable biocompatible polymer. Although suture anchors 38 and 39 are ring-shaped, other shapes (e.g., squares, half-rings, thin plates or “ears” with holes for suture thread) can be employed. In other embodiments, suture anchors may consist of larger diameter rings cut from polymer tubes and attached to the catheter using epoxy, other kinds of glue, or adhesives. In still other embodiments, suture anchors may be manufactured as part of the extrusion process or they may be heat-formed. Alternatively, suture anchors may be bumps on the surface of the tubing made of silicone elastomer, epoxy, or other kinds of adhesives.

The number of suture anchor sets and locations on a catheter may vary, but in at least one embodiment there are two sets of suture anchors located about 3 cm. and 13 cm. from the needle. The number of molded rings at each location is 3 in FIGS. 24 and 25, but can vary from, e.g., about 1 to 5. The distance between each ring in the embodiment of FIGS. 24 and 25 varies from about 0.2 mm and 2 mm (e.g., around 1 mm). The outer diameter of suture anchors 38 and 39 varies from about 0.5 mm to 4 mm (e.g., about 1.4 mm).

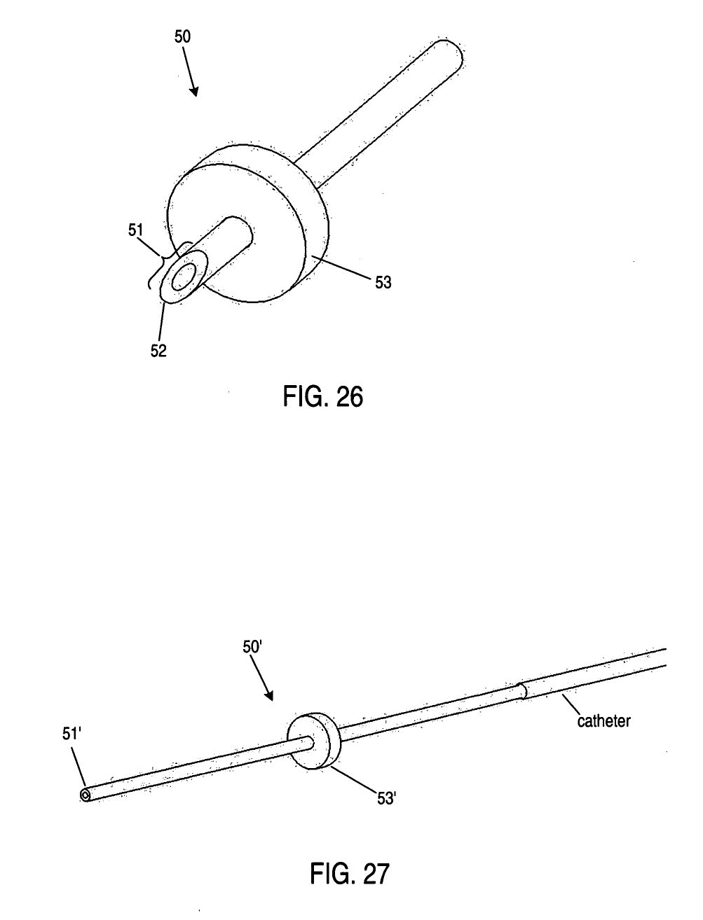

As seen in FIGS. 1-3, a needle 50 is positioned at the distal end of fluid carrying systems 20A-20C. Needle 50 is sized and configured for easy and effective movement within the middle ear, and for performing round window injections. One embodiment of needle 50, shown in FIG. 26, has a length of about 6 mm, a sharpened end 51 on the distal injection end, and an insertion stop 53. The injection end (or a portion thereof) can be beveled to provide sharpened end 51. In at least one embodiment, the sharpened end has a bevel of about 60 degrees. However, other bevel angles could also be used. Insertion stop 53 is sized and shaped to properly position needle 50 within the middle ear, thereby preventing over insertion of the needle within the ear. Insertion stop 53 has a thickness of about 0.5 mm. In other embodiments, the thickness of insertion stop 53 is between about 0.2 mm and about 1 mm. Insertion stop 53 has a diameter of about 1 mm to about 3 mm. Insertion stop 53 is secured to the needle body at a point approximately 0.5 mm to about 1 mm from the most distal tip 52 of sharpened end 51. However, that distance can be changed for various reasons (e.g., accommodate a need for deeper penetration past the round window).

In an alternative embodiment illustrated in FIG. 27, needle 50′ includes a blunt tip 51′ that does not pierce or otherwise puncture the patient. In this embodiment, the angle of the bevel can be varied between about 0 degrees and about 75 degrees. Also, tip 51′ would be sharpened in different ways compared to point 51. The embodiment shown in FIG. 27 can be used with passages through bone surrounding the inner ear, as discussed below. A catheter would be attached to the distal end of needle 50′. In the example of FIG. 27, insertion stop 53′ is approximately 1 cm from tip 51′. Further, insertion stop 53′ could be formed from a porous biocompatible material such as titanium. When placed into a specially prepared well within a bone, the bone may then grow into and over the insertion stop to form a permanent connection.

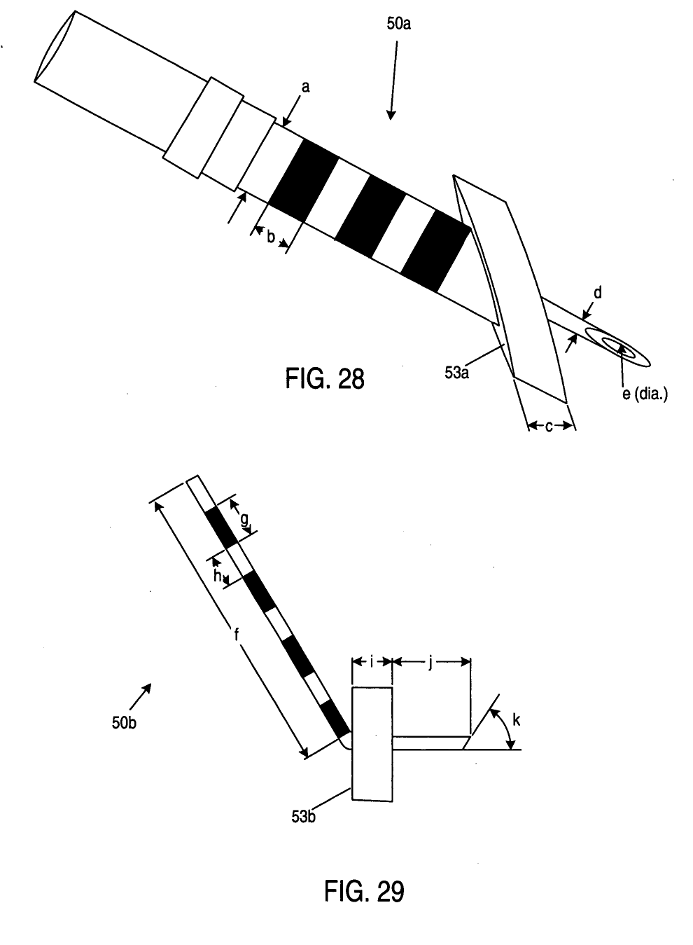

Returning to FIG. 26, insertion stop 53 is positioned along the length of needle 50 to prevent over insertion of needle 50 within the ear. In at least one embodiment, tip 52 of needle 50 is positioned within the scala tympani when the needle is being used. The diameter of insertion stop 53 is sized for positioning the needle in the round window niche and to allow the reproducible insertion and re-insertion later in the same location. The diameter of insertion stop 53 is also sized so as to allow the needle assembly to fit into the round window niche without excessive play in the positioning. Needle 50 can be straight or bent after insertion stop 53 to allow better positioning of needle 50 in the round window. In some embodiments, the angle of the bend is 60 degrees from straight (i.e. 120 degrees; see FIGS. 28-30). Needle 50 is preferably 28 gauge, but can be any convenient size that can penetrate the round window without creating an excessively large hole to be sealed. Needle sizes could be between about 22 gauge and about 35 gauge (e.g., about 28 gauge to about 31 gauge). Insertion stop 53 is welded to the needle shaft using methods known in the art such as laser welding. Alternatively, insertion stop 53 can be attached with epoxy, other kinds of glue or adhesives. Additionally, if the internal hole of the insertion stop is sized correctly, it can be heated to enlarge the hole and a needle (sized correctly for the hole) can be inserted to the correct depth down the shaft. The insertion stop is then allowed to cool and make a tight seal around the needle shaft. This later method would obviate the need for welding the insertion stop and would allow the application of insertion stops to gauges smaller than 31 (as such gauges are difficult to weld). It would be an alternative to, or in addition to, gluing the insertion stop 53 onto the needle shaft. The shaft would be roughed up to enable a tight fit of a catheter tubing with an inside diameter appropriate to the gauge of the needle.

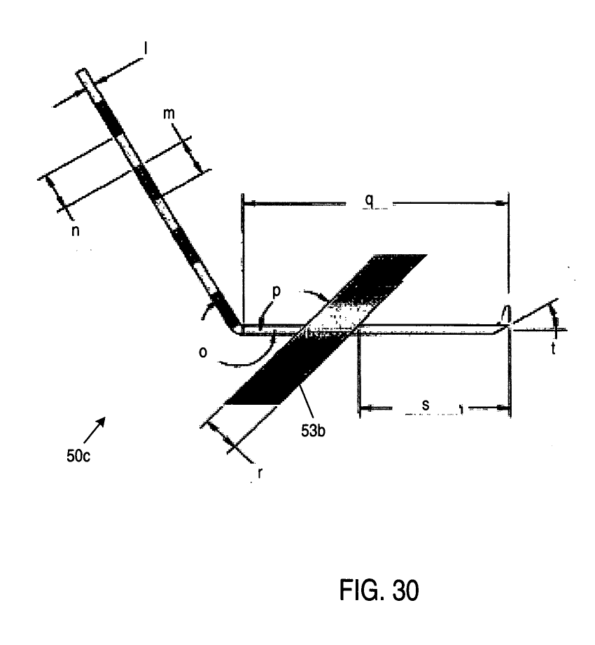

Needles according to additional embodiments are shown in FIGS. 28-30. Needle 50a (FIG. 28) includes a generally elliptical insertion stop 53a. Needle 50b (FIG. 29) includes a generally round insertion stop 53b. The end of the needle having point 51b extends generally perpendicular to insertion stop 53b, with the other end of needle 50b (intended for insertion into a catheter) being at a non-perpendicular angle to insertion stop 53b. Needle 50c (FIG. 30) includes a generally elliptical insertion stop 53c (with the major axis in the plane of the page). Contrasting bands on the needles in FIGS. 28-30 help a physician gage depth. Dimensions for needles 50a, 50b and 50c according to some embodiments are provided in Tables 1-3, respectively, but dimensions may differ in other embodiments.

TABLE 1

| Dimension | Value |
|---|---|
| a | 1.00 mm |
| b | 1.00 mm × 0.50 mm |
| c | 2.00 mm × 0.50 mm |
| d | 0.20 mm |
| e | 0.10 mm |

TABLE 2

| Dimension | Value | |
|---|---|---|
| f | 4 | mm |
| g | 0.50 | mm |
| h | 0.50 | mm |
| i | 0.50 | mm |
| j | 1 | mm |

| | |
|---|---|
| k | 60° |

TABLE 3

| Dimension | Value | |
|---|---|---|
| l | 0.1225 | mm |
| m | 0.50 | mm |
| n | 0.50 | mm |
| o | 120° | |
| p | 135° | |
| q | 3.50 | mm |
| r | 0.50 | mm |
| s | 2.00 | mm |

| | |
|---|---|
| t | 30° |

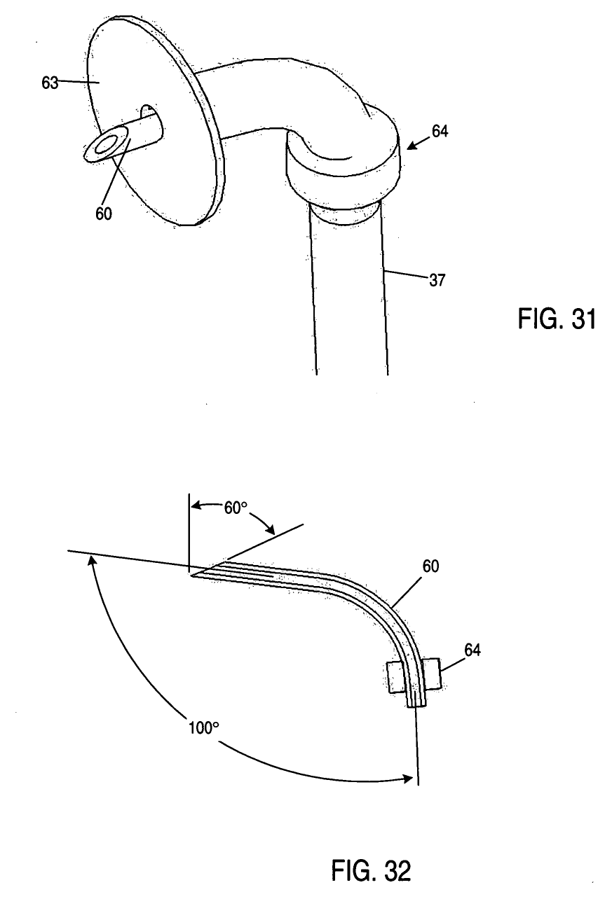

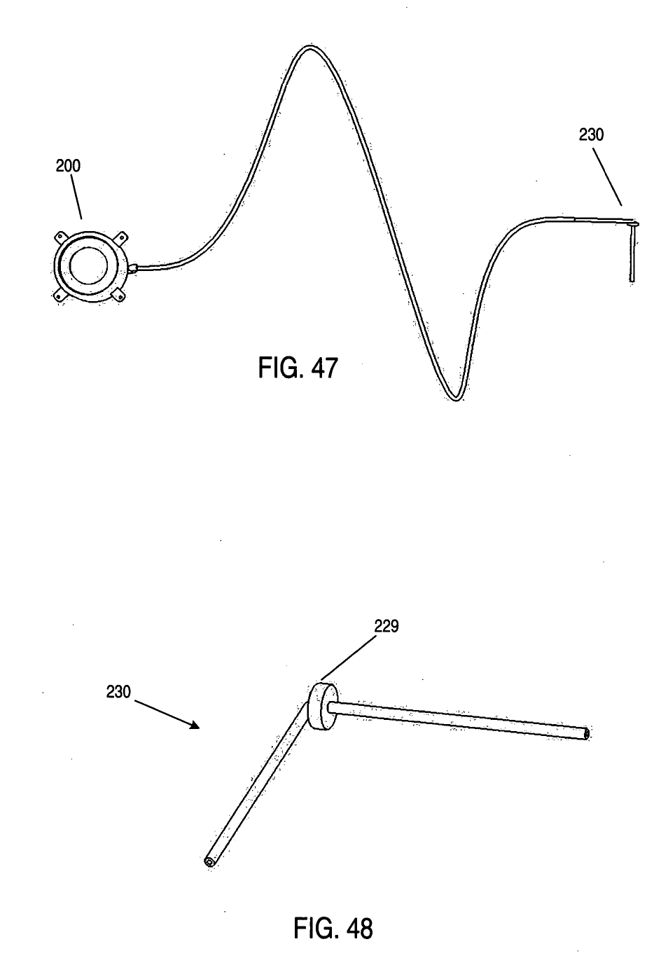

As seen in FIG. 4, a needle 60 is positioned at the end of fluid carrying system 20D. Needle 60 is also sized and configured for easy and effective movement within the middle ear, and for performing round window injections. In alternate embodiments (e.g., as shown in FIG. 47) the needle can be inserted through the bone surrounding the cochlea (thus avoiding the middle ear and maintaining a sterile environment in and around the needle insertion site into the cochlea or neural injection site). FIG. 31 is a perspective view illustrating injection needle 60 according to at least one embodiment. Needle 60 is contained in an end of catheter 37, and extends from an insertion stop 63 for round window injection into the cochlea.

FIG. 32 is a sectional view of needle 60 prior to placement within the end of catheter 37. Needle 60 includes a flange 64 to provide a tight connection within catheter 37 without the need for gluing catheter 37 to needle 60. Flange 64 prevents needle 60 from sliding out of the end of catheter 37. Needle 60 may consist of one whole part, or two separate parts where flange 64 (if metal) is welded to the remainder of the needle. Flange 64 can be welded to the needle shaft using known methods in the art such as laser welding. Alternatively a plastic or metal flange can be attached with epoxy, other kinds of glue or adhesives. Additionally, if the internal hole of a flange is sized correctly, it can be heated to enlarge the hole and a needle shaft (sized correctly for the hole) inserted to the correct depth down the shaft, with the flange then allowed to cool and make a tight seal around the shaft. This method would eliminate the need for welding the flange and would allow the application of flanges to smaller-sized needles where welding might melt a hole in the needle shaft. It would be an alternative, or in addition to, gluing a flange onto a needle shaft. It is not necessary to have a flange on a needle. Moreover, a needle may have one or more flanges, positioned anywhere on the needle, which serve different functions such as strengthening a connection with the catheter tubing or serving as an insertion stop. The needle and flange can be made of 316 stainless steel, titanium, or any other biocompatible metal. Alternatively, a flange may be made of biocompatible, drug compatible polymers/plastics such as fluoropolymers, urethanes, and other thermoplastic elastomers, polyethylenes, nylons, acetals, polycarbonates, and various other plastics. A needle can also be made from a hard plastic that can be bent or molded to allow a specific angle bend and, optionally, contain a molded or glued plastic flange for attachment to a catheter. An advantage of a plastic needle is potentially improved bonding with glue.

In the embodiment of FIG. 31, there is a single flange 64 which acts to strengthen the connection between needle 60 and catheter 37. Flange 64 is located on the needle about 1 mm from the non-beveled (proximal or upstream) end. This distance from the proximal end can vary from about 0.1 mm to 2 mm. Flange 64 has a diameter of about 0.5 mm to 3 mm and a length of approximately 0.2 mm to 3 mm.

In other embodiments, a needle may be flared to a larger diameter at the proximal end, serving a similar purpose as the flange. A needle shaft may also be roughened or primed to allow for a stronger bond between the needle and catheter using epoxies or other glues, obviating a catheter attachment flange in some cases.

The distal (injection) end of the needle 60 is beveled to provide a sharpened point (for embodiments where the device is to be used in round window or other kinds of injections) having an angle of about 60°. In other embodiments, the angle varies from about 10° to 80°. Needle 60 is preferably 28 gauge, but can be any convenient size that will allow penetration of the round window without creating an excessively large hole to be sealed on removal of the needle, and without producing excessive scar tissue to prevent the normal working of the round window membrane. In some other embodiments, the size of the needle varies from about 22 gauge to 35 gauge. The end-to-end length of needle 60 varies from about 3 mm to 10 mm (e.g., around 6 mm).

In the embodiment of FIGS. 31 and 32, needle 60 is curved 100° from the middle to the proximal end of the needle. Other embodiments for other uses may have different needle bending or curving geometries. For example, a needle may be straight, or it may have one or more bends or curves designed for easy movement within the middle ear and easy round window injection, avoidance of the basilar membrane following insertion or insertion through the temporal bone into the cochlea or mastoid bone for other objectives.

In an embodiment in which the injection device (e.g., needle 50′ of FIG. 27 or needle 230 of FIG. 48) is inserted through a bone into the cochlea (e.g., through a hole drilled by a surgeon), the needle can be blunt tipped as well as sharpened, as the hole drilled through the bone removes the requirement to use a sharp tip to penetrate the tissue. In one such embodiment the bone needle can be significantly longer (for example 10 to 30 mm) to allow adequate penetration through the bone. Such a needle can be bent to allow complete implantation below the skin for long term implantation or through the skin for short term usage. Such a needle may have a similar insertion stop and needle to catheter attachment requirements as the needle described above for round window injection applications. For bone needles intended for permanent implantation, a material such as porous titanium is preferred for insertion stop 229.

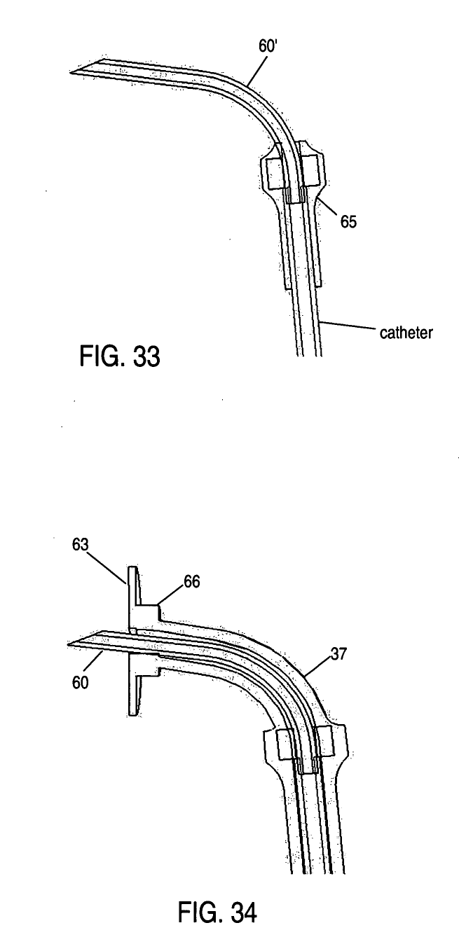

FIG. 33 is a cross-sectional view of another embodiment of a needle 60′ in which heat-shrink tubing 65 is used to provide a firm connection between the needle and the catheter tubing. The biocompatible and drug compatible heat-shrink tubing 65 is made of PTFE; other possible materials include FEP and other fluoropolymers, polyester, polyolefin or other polymers. The expanded inner diameter of the heat-shrink tubing should be larger than the needle flange (e.g., around 0.044″). Recovered inner diameter of the heat-shrink tubing should be small enough to fully encase the flange and catheter tubing as shown in FIG. 33. Length of the heat-shrink tubing can vary from about 3 mm to 10 mm (e.g., about 4 mm).

In another embodiment, the catheter tubing may be bonded directly to the needle shaft using epoxy, or other kinds of glue or adhesives.

Catheter tubing can be attached directly to the needle shaft solely as described previously, or in conjunction with the heat-shrink tubing connection. The catheter can be glued or attached to the needle barrel using epoxy type glues or other methods common in the art to attach plastic to metals. The positioning of a metal or plastic flange to the proximal end of the needle around which the tubing can be attached makes a very strong attachment.

In at least some embodiments, an insertion stop is included to prevent over-insertion of the needle within the ear. The insertion stop is sized and shaped to properly position the needle in the round window niche, and to allow the reproducible insertion and re-insertion later in the same location. The diameter of the insertion stop is also sized so as to allow the needle assembly to fit into the round window niche without too much play in the positioning.

FIG. 34 is a cross-sectional view of the complete needle assembly from FIG. 31, including the outer tubing of catheter 37 and insertion stop 63. The outer tubing of catheter 37 can be made of a flexible, biocompatible, drug compatible polymer, preferably silicone rubber, which expands when exposed to certain solvents. The insertion stop 63 is made of silicone rubber sheeting, but could also be made of polyester mesh, nylon mesh or any biocompatible polymer sheeting or mesh. Diameter of insertion stop 63 varies from about 1 mm to 4 mm (e.g., about 3 mm). Insertion stop 63 may be directly attached to the flexible tubing or may be bonded to a flange 66, which is molded into the end of the catheter 37. In the embodiments of FIGS. 31 and 34, the insertion stop is about 1.5 mm from the beveled needle injection tip. In other embodiments, the insertion stop is between about 0.5 mm and 2.0 mm from the beveled needle injection tip. The distance from the distal insertion end (in one embodiment the beveled point) can be changed to accommodate the need for deeper penetration through the round window and into the cochlea. Further, insertion stop 63 is preferably transparent and flexible for easier positioning and observations within the round window niche or in other compartments, but a rigid plastic or metal flange or non-transparent flange may be used. A flexible insertion stop may operate to secure the needle in place once the round window has been penetrated by the needle tip. Specifically, the insertion stop bows slightly and is mildly wedged into the round window niche.

In another embodiment, an insertion stop may be molded directly to the outer catheter tubing using an acceptable biocompatible polymer, such as silicone elastomer. Alternatively, an insertion stop may consist of a larger diameter slice of flexible tubing (e.g., silicone), that is bonded to the outer catheter tubing using epoxy or other kinds of glue or adhesives, such as silicone adhesive. In yet another alternative embodiment, the insertion stop may be formed by heating the tip of the outer catheter tubing, and flaring or shaping it into the desired size and geometry. In further embodiments, an insertion stop is secured to the needle body, with the insertion stop made of 316 stainless steel, titanium, or any other biocompatible metal. Alternatively, an insertion stop may be made of biocompatible polymers/plastics such as fluoropolymers, urethanes, and other thermoplastic elastomers, polyethylenes, nylons, acetals, polycarbonates, and various other polymers.

In some embodiments insertion stop 63 has a thickness of about 0.5 mm. In other embodiments, the thickness of insertion stop 63 is between about 0.2 mm and about 1 mm. In at least some embodiments, insertion stop 63 has a diameter of about 1 mm to about 3 mm.

A needle assembly can also be provided without an insertion stop. In such embodiments the needle may also be marked with bands (either painted or etched onto the surface) to indicate to the physician how deeply the needle has been inserted.

Returning to FIGS. 1-3, catheters 21-23, 29 and 32 are formed from tubing that is relatively thick walled, with at least one small inner lumen for drug (or other agent) delivery. The tubing is glued or otherwise securely attached to a female luer attachment (e.g., an attachment such as connector 33 or connector 16) or the inlet of an in-line micro-infusion filter (e.g., filter 24 or filter 25). The tubing should be formed of a material that will be compatible with the formulated drug or other agent to be delivered. If the tubing is a multi-component material with a different outer layer as a sleeve over an inner tubing, the inner tubing can be formed of a material (such as Teflon) that is drug compatible and the outer sleeve made from a material that can be secured to one or all of the filter(s) and the quick disconnect 28. The internal lumen of the tubing has a diameter large enough to allow the delivery of the desired amount of therapeutic agent(s) to needle 50 without excessive back pressure from the tubing and filter assembly. The outer diameter of the tubing should be approximately the same size as the inside diameter of the downstream end of the housing for connector 16 (or of another appropriate connector) and an upstream end of the quick disconnect 28 or an upstream end (inlet) of the in-line micro-infusion filter 24 (antibacterial filter). A catheter (e.g., catheter 21) can have a single or multiple lumens.

Catheter 29 forms a portion of fluid carrying systems 20A and 20B and to needle 50. Like catheter 21, catheter 29 is chemically inert, flexible and biocompatible. Catheter 29 is very small tubing that has an outer diameter sized for convenient insertion into the middle ear and an inner diameter that allows it to receive and hold round window injection needle 50. Catheter 29 can be made from a perfluoro hydrocarbon (e.g., PTFE or FEP), although other chemically resistant tubing (such as polyethylene, polypropylene, and polyamide) could be used. The tubing of catheter 29 could also be flanged at one end to help anchor catheter 29 to the outlet of micro-infusion filter 25. Catheter 29 does not need a flanged end, when, for example, the bonding surface is roughened to make a bonding surface with the connecting tubing placed inside the micro-infusion filter to help hold the catheter in place.

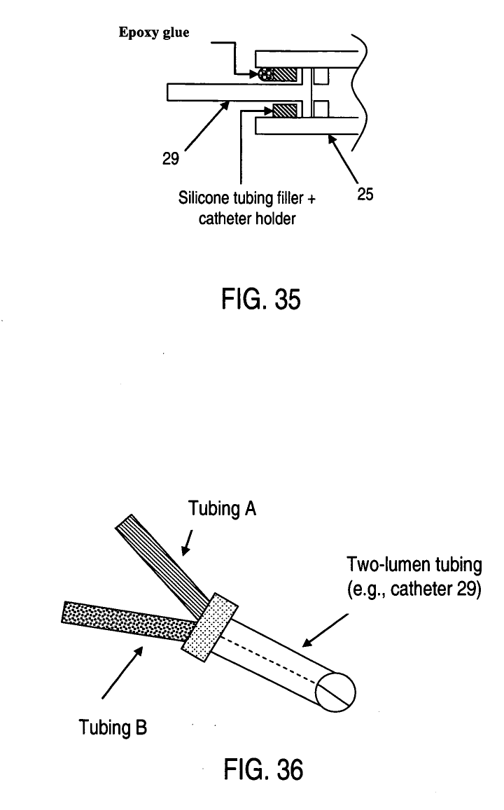

FIG. 35 (a cross-sectional view) illustrates how a flanged end of catheter 29 can be used to prevent catheter 29 from separating from other parts of apparatus 10A or 10B, such as the inlet and/or outlet of micro-infusion filter 25. The micro-infusion filter inlet and/or outlet tubing is assembled in the illustrated embodiment to securely retain the flanged end of catheter 29. The illustration shows a flanged catheter 29, but non-flanged tubing for catheter 29 can also be used. An epoxy, glue or other type of bonding agent can be used to hold the silicone tubing filler in place which in turn holds catheter 29 in place. In the embodiment illustrated in FIG. 35, the bonding agent can include Epoxy Ablestic—National Starch & Chemical Co.; Abelux: HGA-3U; Cage 21109; Batch: 5084 998. The steps of securing catheter 29 to the filter assembly include: first, with the tubing of catheter 29 already inserted into the filter inlet/outlet, advancing the silicone and PTFE tubing as far as possible in the filter's inlet/outlet; second, preparing epoxy in a syringe with a proper luer tip; third, filling the space between the PTFE tubing and the inlet of the filter with epoxy, and curing with UV light to fix the connection; and fourth, verifying the strength of the connection by pulling the filter and the tubing in different directions and inspecting the connection under a microscope.

Multi-lumen tubing can also be used. In some embodiments, use of multi-lumen tubing allows for the separate or simultaneous delivery of multiple drugs, solutions or other therapeutic agents at the same or different delivery rates within the inner ear. Examples of multi-lumen tubing include tubing having two, three or four inner lumens. The lumens of the multi-lumen tubing can be concentric, side-by-side or a combination of both. FIG. 36 illustrates an example of utilizing a double lumen tubing for a catheter such as catheter 29 (see FIGS. 1 and 2), but with two separate inputs (tubing A and tubing B). Tubing A could, e.g., be in fluid communication with an anti-bacterial filter, a quick disconnect coupling, an additional catheter and a syringe (e.g., all of the components upstream of catheter 29 in FIG. 1 or FIG. 2). Tubing B could be, e.g., in fluid communication with a separate anti-bacterial filter, quick disconnect coupling, additional catheter and syringe. In other embodiments, tubing A and/or tubing B could have other types of inputs (some of which are provided as examples below).

An advantage of using multi-lumen tubing is the compact nature of the tubing that allows one tube to be inserted through the ear canal and into the inner ear that is capable of delivering multiple solutions. At one end of a multi-lumen tubing, the different inputs can be attached to the appropriate hole(s) to receive the respective therapeutic (or other type) agent(s) or source of negative pressure. The other end can be attached to a section of elongated tubing to mix the individual inputs before delivering the final solution of agents to the needle. As another example, multi-lumen tubing could also be used to deliver a solution in one lumen while withdrawing a sample through another lumen. As yet another example, one of the lumens in a multi-lumen tubing could be used to provide access for a wire or other element into an inner ear as a sensor or stimulator. As still another example, a lumen of a multi-lumen tubing could be used to deliver a conductive solution into an inner ear or other anatomical region, with the conductive solution then used to send and receive signals from a target region.

In an embodiment using a four lumen tubing (not shown), one elongated channel could be used to inflate a balloon inside the inner ear, which balloon is capable of holding a dialysis or delivery membrane against a specific tissue. A second channel could be used to deflate the balloon. A third channel could be used to deliver a therapeutic solution to the membrane, and the fourth channel could be used to withdraw the spent therapeutic solution or withdraw a sample from the area, for example, to test the effectiveness of the drug delivery. In a two lumen tubing one lumen can deliver a solution containing a concentrated therapeutic in a vehicle promoting stability and solubility while delivering in a second lumen a diluting vehicle to be mixed with the concentrated therapeutic to produce the proper formulation for delivery to the target tissue. A mixing chamber can be positioned (e.g., at or near a terminal end of the two lumen tubing) to mix two or more different solutions prior to delivery of the mixture into an inner ear or other animal tissue. A needle for injecting the final formulation into the target tissue, such as needle 50, can also be secured to the end of the multi-lumen tube.

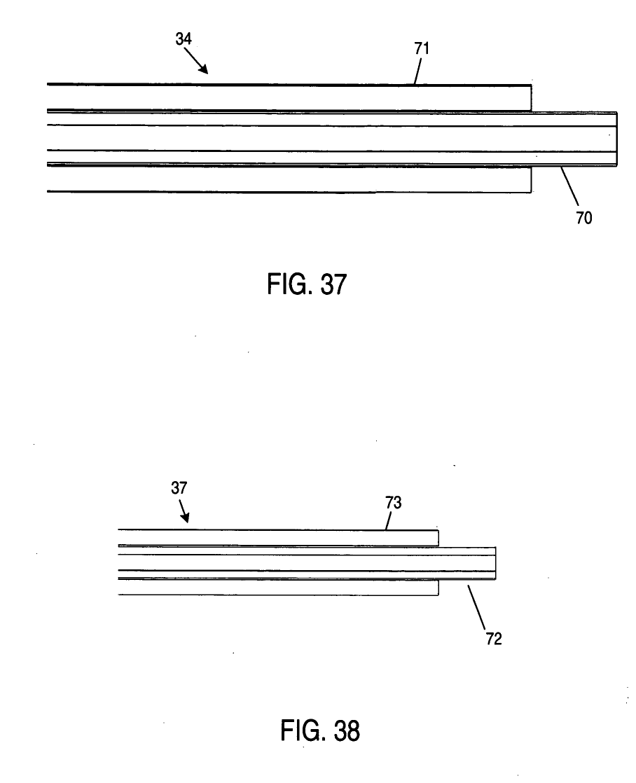

FIGS. 37 and 38 show, respectively, cross-sectional views of catheters 34 and 37 in FIG. 4. The catheters shown in FIGS. 37 and 38 could also be used in other embodiments (including the embodiments of FIGS. 1-3). Catheters 34 and 37 are both relatively thick walled with at least one small inner lumen for delivery of a drug or other agent. The tubing surface in contact with the fluid flow is formed of a material that will be compatible with the formulated drug or other therapeutic agent to be delivered. The internal lumens have diameters large enough to allow the delivery of the desired amount of therapeutic agent(s) to the needle without excessive back pressure from the tubing and filter assembly. Catheters 34 and 37 can have single or multiple lumens. In at least one embodiment, the tubing is partially transparent, allowing a person to view fluid flow, bubbles, or blockages in the tubing.

Catheter 34 extends from luer 33 to quick disconnect 28, inline filter 36, or catheter 37. In the embodiment of FIG. 4, catheter 34 extends between luer 33 and quick disconnect 28, and a catheter similar to catheter 34 connects quick disconnect 28 and filter assembly 36. The tubing of catheter 34 may consist of one or more layers of materials, each selected to provide certain beneficial qualities and characteristics such as biocompatibility, drug compatibility, flexibility, strength, kink-resistance, or connection capabilities as well as resistance to water permeability, CO₂ and other environmental chemical permeabilities.

In the embodiment of FIG. 37, catheter 34 includes two layers. The inner layer 70 consists of tubing which is made of a biocompatible and relatively chemically inert material, such as polyimide or a fluoropolymer (e.g., PTFE). The outer layer 71 consists of tubing which is made of a flexible, biocompatible polymer (e.g., silicone rubber). Alternately, outer layer 71 can be made of polyurethane, polyvinylchloride (PVC), polyethylene, vinyl, or other flexible, biocompatible polymers. Inner layer 70 may be inserted into outer layer 71 after the expansion of outer layer 71 with solvents. When the solvent(s) evaporates, the outer layer returns to its design diameter and closes around the inner layer to make a tight seal between the two layers. The inner and outer layers then adhere together directly because of frictional or self adhesive properties of these layers. In further embodiments, two or more layers can be adhered together with the use of curing, heating, adhesives, or other suitable bonding techniques. For example, an intermediate layer between the inner and outer layers may include an adhesive.

In still other embodiments, multiple layered tubing can be manufactured using other methods known in the art, such as co-extrusion. Co-extrusion can simplify and expedite the manufacturing process and allow the tubing to be made economically and efficiently. In yet other embodiments, the layers may be formed by other manufacturing techniques, including, but not limited to molding, layering sheets and rolling, or the like.

The inner diameter of layer 71 may be approximately the same size as the outside diameter of the downstream end of luer 33 and an upstream end of quick disconnect 28. Non-limiting examples of dimensions for catheter 34 include: inner diameter of inner layer 70 between about 0.010 inches and about 0.030 inches (e.g., about 0.018 inches) with a thickness of about 0.004 inches to about 0.018 inches (e.g., about 0.009 inches); outer layer 71 thickness between about 0.010 inches and 0.045 inches (e.g., about 0.030 inches).

To increase bonding capability, the catheter tubing surfaces may be treated using methods known in the art, such as priming, etching, or surface roughening. Thus the catheter can be attached to the luer 33, quick disconnect 28, filter assembly 36, or catheter 37 using adhesive bonding, solvent bonding, clamping, flanging, ultrasonic welding, or the like.

Returning to FIG. 4, catheter 37 extends from inline filter 36 to needle 60. In other embodiments, catheter 37 may be directly connected to quick disconnect 28 or to catheter 34. The tubing of catheter 37 may consist of one or more layers of materials, each selected to provide certain beneficial qualities and characteristics such as biocompatibility, drug compatibility, flexibility, strength or connection capabilities. Catheter 37 is a very small tubing that has an outer diameter sized for convenient insertion into the middle ear and an inner diameter that allows it to receive and hold round window injection needle 60.

FIG. 38 is a sectional view of catheter 37 according to at least some embodiments, and shows an inner layer 72 and an outer layer 73. Inner layer 72 consists of tubing made of a biocompatible, drug compatible and relatively chemically inert material, such as polyimide or a fluoropolymer (e.g., PTFE). Outer layer 73 consists of tubing which is made of a flexible, biocompatible polymer (e.g., silicone rubber). Alternately, the inner and/or outer layers can be made of polyurethane, polyvinylchloride (PVC), polyethylene, vinyl, or other flexible, biocompatible polymers. Inner layer 72 may be inserted into outer layer 73 after outer layer 73 has been expanded using solvents. When the solvent(s) evaporate, outer layer 73 returns to its design diameter and closes around inner layer 72 to form a tight seal between the layers. As with catheter 34, alternate embodiments of catheter 37 could include more than two layers (e.g., and adhesive layer between layers 72 and 73).

Non-limiting examples of dimensions for catheter 37 are as follows: the inner diameter of inner layer 72 may be between about 0.006 inches and about 0.020 inches (e.g., about 0.010 inches), with a wall thickness between about 0.004 inches and about 0.018 inches (e.g., about 0.008 inches); the wall thickness of outer layer 73 may be between about 0.008 inches and 0.030 inches (e.g., about 0.015 inches).

In other embodiments, multiple layered tubing for catheter 37 can be manufactured using other methods known in the art, such as co-extrusion.

In at least some embodiments, the inner layer 70 of a portion of catheter 34 between quick disconnect 28 and filter assembly 36 would take the place of tubing 144 in FIGS. 15-19, with the inner layer 72 of catheter 37 serving as tubing 145. In other words, filter assembly 36 may be formed directly onto the inner layers of the catheters to which it is connected, with tubings 144 and 145 placed into the unflared ends of metal connectors 141 and 142 being the inner liners from the catheters.

Although FIGS. 37 and 38 show catheters having two layers, catheters in other embodiments may have a single layer or may (as previously indicated) have more than two layers.

In at least one embodiment, the catheter tubing 34 is attached to a syringe via female luer tip 33 that cooperates (e.g., locks) with a male luer tip (or other appropriate connector) at the downstream end of the syringe. Female luer tip 33 has a standard size that enables easy connection to the male tip. In such an embodiment, the resulting interface between the catheter and the syringe would be a simple disconnection. In an alternative embodiment, the infusion set could have the catheter connected directly to the syringe and attached by an appropriate glue. This would provide less opportunity for a sterility break within the infusion set. However, this arrangement would make it more difficult to load the syringe.

In at least some additional embodiments, a subcutaneous port is used to supply a drug or other agent to a needle implanted into a patient's cochlea or other location. A subcutaneous port (which may include an attached filter) is connected to a catheter; the catheter then carries an agent from a reservoir in the port to a needle located at the site where the agent is to be applied. In this manner, a subcutaneous port provides a convenient method to repeatedly deliver medication, parental solutions, blood products, and other fluids to numerous tissues for a variety of purposes, and without utilizing significant surgical procedures at each time of delivery. As one example, a subcutaneous port could be placed on the side of the skull (e.g., the mastoid bone) and the catheter extended to the cochlea to deliver a drug or other agent into the cochlea. As another example, a subcutaneous port installed on the mastoid bone (or at another location) could be used to deliver a drug or other agent to a specific location within the brain. Once the subcutaneous port is implanted, a physician can place a drug or other agent within the port reservoir by injecting the agent through the patient's skin and into the port. The agent would then be delivered from the port (via a catheter) to the cochlea, brain or other desired region.

In some embodiments, a port is only partially implanted. In other words, a portion of the port extends through a hole in the patient's skin and is exposed. Such a port allows a physician to inject an agent into the port without having to pierce a patient's skin, thereby avoiding patient discomfort and potential contamination of the agent with the patient's own blood. Partially implanted ports also have potential disadvantages, however. In particular, protrusion of the port through the skin can increase risk of infection. However, recently developed technology allows construction of ports using materials that permit a patient's skin to grow into (and bond with) especially prepared device surfaces. In this manner, a more sterile and germ-tight connection between the port and the skin is possible.

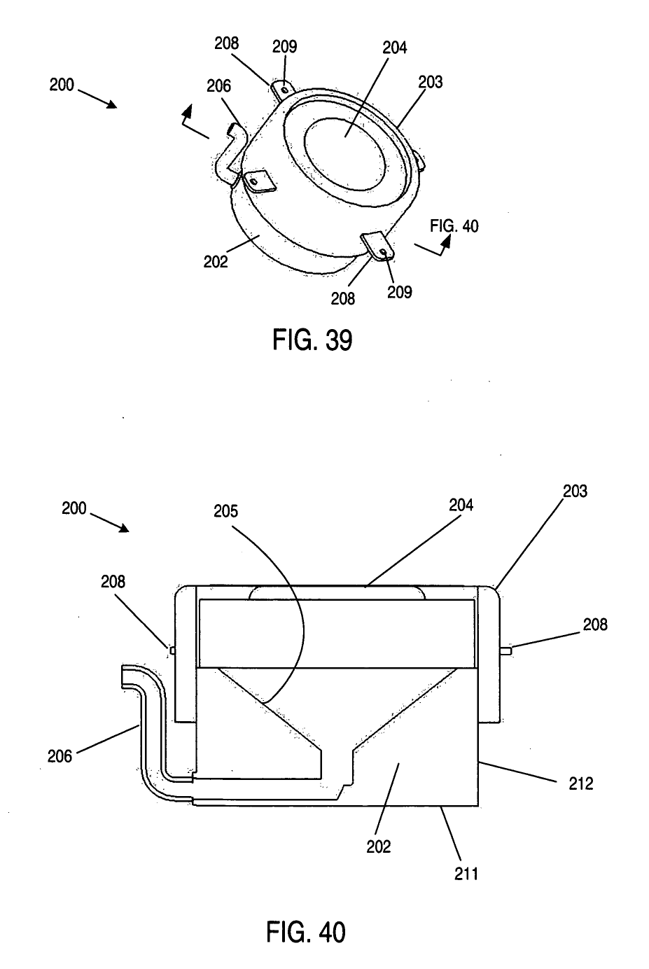

FIG. 39 shows a port 200 according to at least some embodiments. Port 200 includes a reservoir 202 and a cap 203. Cap 203 includes a self-sealing septum 204. Septum 204 is formed from, e.g., a silicone elastomer. Reservoir 202 includes an internal cavity 205 (not shown in FIG. 39, but discussed in more detail below) that is accessible via septum 204. Cavity 205 is also in fluid communication with an outlet 206. Outlet 206 includes, or is attached to, a catheter (not shown) for accessing a vein or other body part (e.g., a cochlea, a brain region, etc.). In the embodiment of FIG. 39, cavity 205 of port 200 has a low internal volume so as to minimize the dead volume of the system. Two or more ears 208 (each having a screw hole 209) extend from cap 203. The purpose of ears 208 is described below.

FIG. 40 is a cross-sectional view of port 200 from the location shown in FIG. 39. Reservoir 202 has a cylindrically shaped outer wall 212 and a conically shaped inner wall forming cavity 205. The conical inner wall of cavity 205 reduces the void volume of port 200. The conical shape also acts to guide a percutaneous needle to the bottom of cavity 205. In other embodiments, internal cavity 205 may be cylindrical or of some other shape. Cap 203 can be made from metal (e.g., titanium or stainless steel), polysulfone or other suitable biocompatible plastic. In at least some embodiments, the height of port 200 is between about 5 and 10 mm (e.g., about 6 to 8 mm), with the diameter of port 200 between being about 10 mm (e.g., 8 mm). These dimensions permit port 200 (after installation) to be palpated through the skin, but do not cause port 200 to protrude so far as to cause irritation to the patient during sleeping.

When installed, port 200 may be placed in a depression that is drilled or otherwise formed in the patient's skull or other bone. Port 200 is then secured in place with self-tapping bone screws placed through holes 209 in ears 208. Ears 208 and holes 209 are positioned sufficiently away from the port body so that the self tapping bone screws do not crack the bone adjacent to the newly created port depression. In at least some embodiments, a port has cylindrical exterior walls at least from the equatorial ring to the bottom. Septum 204, is positioned over cavity 205 and is sealed over the cavity 205 by cap 203. Septum 204 is in some embodiments a wafer-like cylindrical block of silicone, or may be premolded to other shapes. In at least some embodiments the septum includes a flanged region, and the reservoir presses tightly against the flanged region to make a tight fluid- and antibacterial-resistant seal. The bottom surface of septum 204 facing cavity 205 may be undulated in shape (to, e.g., further reduce cavity volume). In at least some embodiments, septum 204 is held onto reservoir 202 by cap 203, with cap 203 mechanically secured to reservoir 202 as described below. In alternate embodiments, a septum may be adhesively attached to a port cap. In still other embodiments, a septum may be attached to a cap by means of a force fit or other mechanical means.

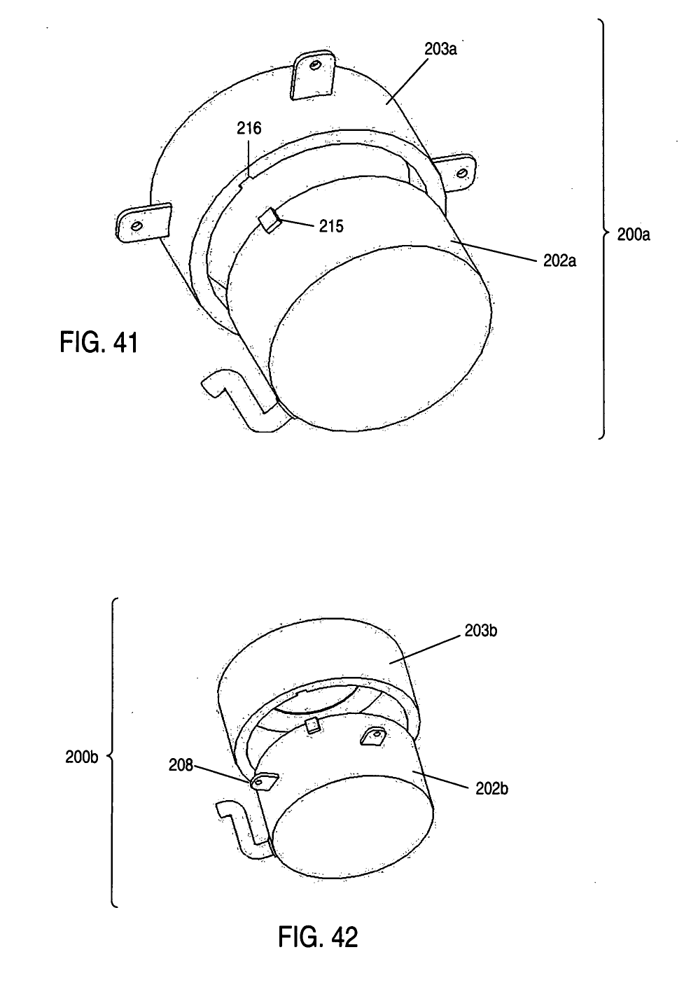

Reservoir 202 is in some embodiments formed from metal (e.g., titanium or stainless steel). Reservoir 202 includes a bottom 211 and a continuous sidewall 212. The diameter of sidewall 212 is slightly greater than the inner diameter of cap 203, which allows reservoir 202 to fit tightly and snap into place inside cap 203 during assembly. Reservoir 202 may include an annular groove positioned on its sidewall, which groove may be compatible with an extruded ring in cap 203, thus allowing reservoir 202 to lock in place. In another embodiment shown in FIG. 41 (port 200a), reservoir 202a includes extruded tabs 215 that slide into associated longitudinal slots 216 in cap 203a, and that can be twisted to lock in place. In yet other embodiments the reservoir and the housing may have mating threads so that the housing and the reservoir can be screwed together. Many of the above-mentioned embodiments provide flexibility for a physician by facilitating changing of the septum (e.g., if the septum begins to leak or becomes loose or loses integrity from repeated punctures, or to replace an internal anti-bacterial filter).

Ears 208 are located on port 200 at a level appropriate for attaching the port to bone. In at least some embodiments, ears 208 are at a level on the sides of cap 203 such that the undersides of ears 208 (i.e., the sides opposite the sides shown in FIG. 39) will rest on bone when the port is installed into a depression of a predetermined depth. Ears 208 can either be attached to cap 203 (e.g., by welding) or molded as a part of cap 203. In alternate embodiments, and as shown in FIG. 42 (port 200b), ears 208 can be located on a reservoir 202b instead of on a cap 203b.

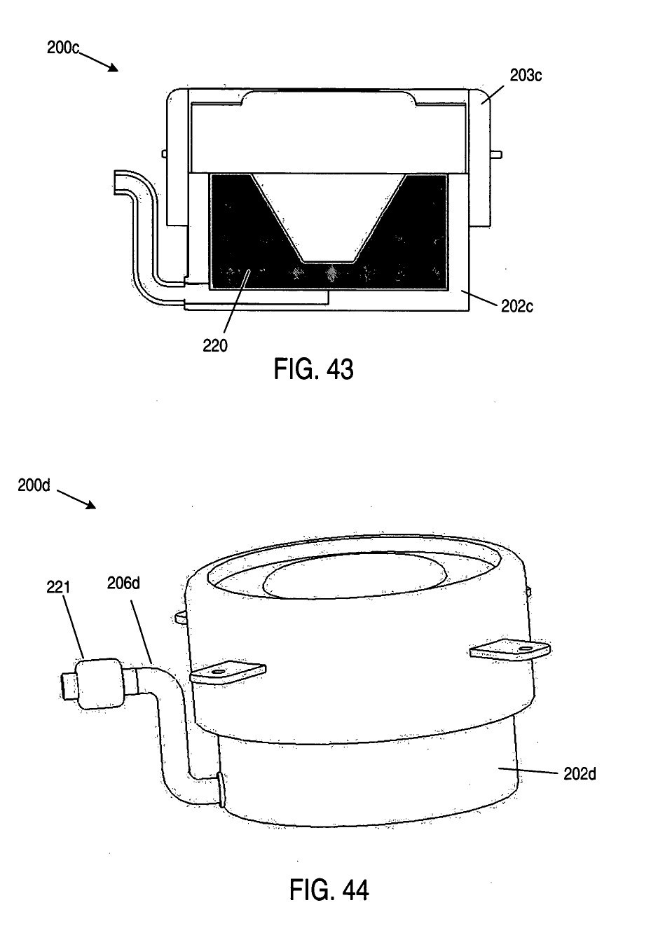

In at least some embodiments, and as shown in FIG. 43 (a cross-sectional view from a location similar to that used for FIG. 40), a port 200c has a reservoir 202c that includes a 3-D porous metal (e.g., titanium or stainless steel) antibacterial filter 220. Such a filter helps provide sterility to the fluid that is introduced into the port after it is implanted in a patient. Filter 220 is positioned so that all delivered liquid (drug and vehicle) passes through the filter to ensure sterility of the port outflow. The dead volume of filter 220 is reduced to a minimum. Other shapes for filters could also be used. A filter such as filter 220 can also be built into the reservoir and be changeable. In another embodiment shown in FIG. 44 (port 200d), an antibacterial filter 221 is placed outside of a reservoir 202d. Filter 221 is located in a housing connected to outlet tube 206d on one side and to a catheter (not shown) on the other side. This arrangement provides flexibility to a physician to change the filter if it might be clogged.

The outlet tube (e.g., tube 206 of FIG. 39) may have different forms in different embodiments. In some embodiments the reservoir has a horizontal outlet on one side. In other embodiments, the reservoir has an angled outlet (e.g., 45°), a Z- or S-shaped outlet, or a groove on the side of the reservoir where the outlet tube can be released. The outlet tube assembly connects with the catheter (not shown) which is placed within the patient. The catheter can be placed in the patient using any of a number of standard techniques. For example, the catheter is often routed between the skin and the bone or placed in a groove on the bone surface to ensure the skin pressure does not collapse the catheter. The outlet tube and the catheter can be connected in many different ways. For example, the outlet tube can have a hose barb or flange and the catheter tubing can be connected by solvent bonding. The present invention is not limited to any particular type of outlet tube assembly.

Generally, the port is implanted within the body and the catheter is routed to a remote area where the fluid is to be delivered. To deliver the fluid, the physician locates the septum of the port by palpation of the patient's skin. The port access is accomplished by percutaneously inserting a needle, typically a non-coring needle, perpendicularly through the septum of the port and into the reservoir. The drug or other agent is administered by bolus injection or continuous infusion. The fluid flows through the reservoir and an antibacterial filter into a catheter to the site where administration is desired. The ports described herein may be used with catheters and needles described above, as well as with catheters, needles and other delivery devices (e.g., a cochlear implant electrode) described below.

As indicated above, a port may be implanted to the mastoid, temporal or other appropriate bone by making a bed for the port; the port is partially submerged in the skull in a depression carved by the surgeon. The depth of the depression may be approximately 3 mm (depending on the bone thickness). In one embodiment, the screw-hole ring (e.g., the undersides of ears 208) will rest flat against the skull once the lower portion of the port is inserted into the depression. In lieu of ears 208, a port may have a metal or plastic ring around the middle of the port exterior through which screws can pass and enable the surgeon to screw the port to the skull. In some embodiments a port includes two screw holes; other embodiments include 3 or 4 screw holes.

The catheter can deliver medication from a port to a cochlea or other region in many ways. The catheter may be connected with an injection needle (e.g., the embodiment of FIG. 47) through the bone into the cochlea (bypassing the middle ear cavity), permanently sealing the needle to the bone and closing the hole to prevent leakage of perilymph and infections within the cochlea. In another embodiment the catheter may be connected with a cochlear implant electrode (as described below). The treatment agents introduced into the port are released from the cochlear implant electrode through drug delivery holes positioned on the electrode placed inside the cochlea (within the inner ear). The catheter can include multiple lumens.

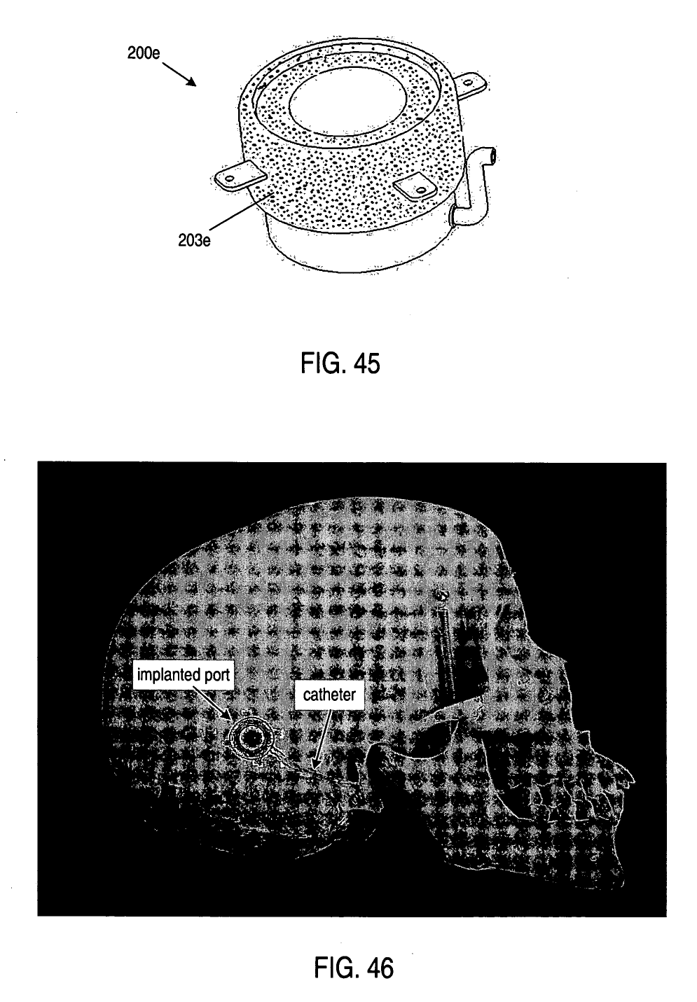

For partially-implanted ports, the reservoir may be placed in the bone bed hole, with the cap partially trans-cutaneous through a hole in the skin (mainly the septum and port top is protruding through the skin) and partially subcutaneous. The port is still screwed to the bone to add stability to the port, but the cap is made from a special material to allow firm attachment of the skin and fibroblasts to the port cover. As shown in FIG. 45, cap 203e of partially implantable port 200e is made of porous biocompatible material (polymeric or metal) which has a surface coating of biomaterials that allow binding of cells to the cap surface. Examples of such coating materials include, but are not limited to, extracellular matrices such as collagen (various types), laminin, glycosoaminoglycan, fibronectin and fibronectin fragments such as peptides that contain the ArgGlyAsp epitope for cell adhesion. The biopolymers and peptides are covalently attached to the material surfaces to ensure the skin cells, including but not limited to the fibroblasts and other cell components to the epidermis, endodermis and the antibacterial layer called the stratum cornium, make a tight bond to the port surface. The porous nature of port cap 203e allows the cells to grow into the port to further ensure that there is a tight connection between the port and the skin. The biopolymers and peptides could also be attached to the cover surface through a hydrogel or hydropolymer. The hydrogel or hydropolymer is attached on one end to the cover surface and the other end to the appropriate biopolymer. The hydrogel or hydropolymer acts like a linker between the surface (whether, plastic, metal or other material) to be integrated into the tissue and the biopolymer and peptides to which the tissue cells bind. The hydropolymer ensures that the cell surface can make appropriate adhesion to the cover surface by putting a spacer between the cover surface and the cell surface.

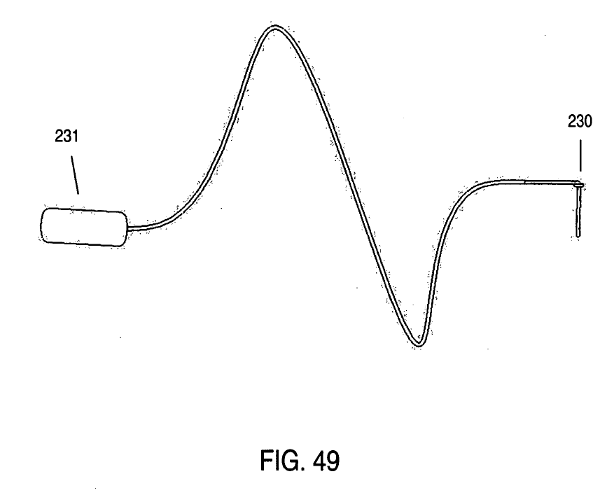

FIG. 46 illustrates a possible location for a port 200 on a patient skull. FIG. 47 shows a port 200 connected to a bone needle 230. FIG. 48 is a drawing of bone needle 230. FIG. 49 is a drawing of bone needle 230 connected to an implantable osmotic pump 231 (such as those available from Durect Corporation of Cupertino, Calif. under the trade name DUROS). In at least some embodiments, the insertion stops on bone needles such as are shown in FIGS. 48 and 27 (as well as bone needles of other configurations) are formed from one or more biocompatible porous materials such as titanium. The porous material may be coated with a bone growth factor such as OP-1. After surgical implantation of the bone needle, the insertion stop becomes fused to (or otherwise integrated into) the bone to form a permanent connection.

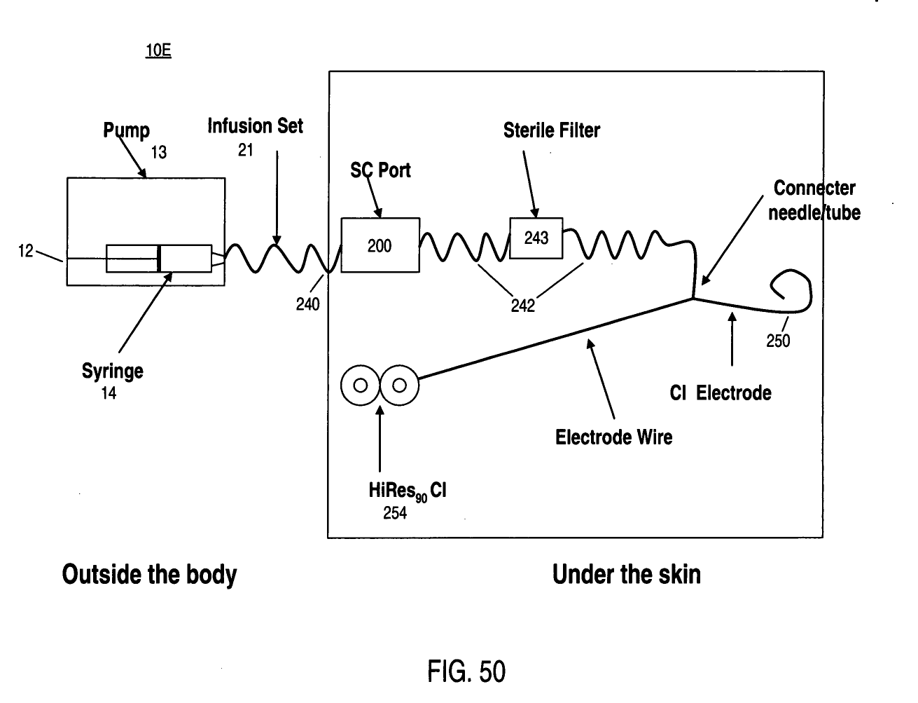

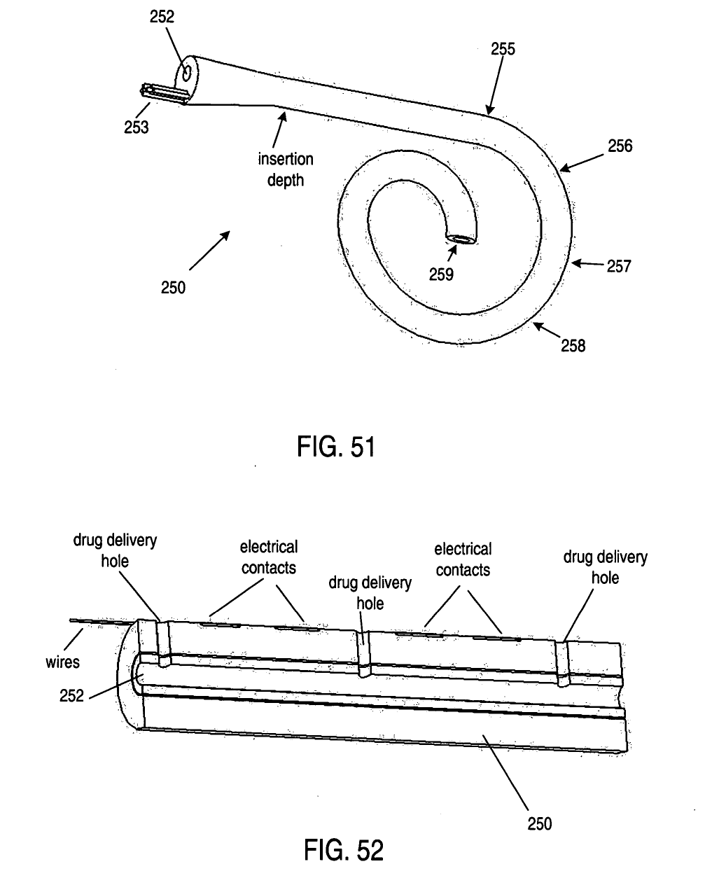

FIG. 50 is a schematic diagram of an apparatus 10E according to at least some additional embodiments. Apparatus 10E includes a portion that is implanted within the body of the patient and a portion that remains outside the patient. Supply system 12, including pump 13 and syringe 14, remains outside the body. Catheter 21 is also located outside the body. As with embodiments described above, supply system 12 and catheter 21 could include one or more of the above-discussed antibacterial (sterilization) filters. The terminal end of catheter 21 includes an injection needle 240 that is introduced into an implanted, subcutaneous port 200. An implanted catheter 242, similar to catheter 29, extends from port 200 and is connected to a cochlear implanted (CI) electrode 250. Catheter 242 can carry one or more sterility filters 243. The treatment agent(s) introduced into port 200 are released from the CI electrode 250 through drug delivery holes 255-259 positioned inside the inner ear (described in more detail below in conjunction with FIGS. 51 and 52). Electrode 250 can include multiple lumens such as that disclosed in U.S. Pat. No. 6,309,410, which is incorporated herein by reference.

As shown in FIG. 50, a cochlear implant electrode could be a component of a system implanted to assist persons with hearing loss (such as, e.g., the HIRES 90 cochlear implant available from Advanced Bionics of Sylmar, Calif.). FIG. 51 is a partially schematic drawing of a cochlear implant electrode 250 according to at least some embodiments. Once placed in a patient's cochlea, implant 250 would have a shape similar to that shown so as to contour to the cochlea. Once implant 250 is in place, electrical contacts (not shown in FIG. 51) on the outer surface of electrode 250 receive signals via wires 253 from electronics 254 (see FIG. 50) and stimulate the cochlea. Because it is located within the cochlea, however, electrode 250 can also be used to deliver drugs or other agents. In particular, a catheter connected to stylet hole 252 can deliver drugs into a duct within electrode 250. Those drugs are then released at locations 255-259. In at least some embodiments, location 258 is approximately 9 mm from location 259, location 257 is approximately 11 mm from location 259, location 256 is approximately 13 mm from location 259, and location 255 is approximately 15 mm from location 259, with the length of electrode 250 being approximately 23.5 mm. FIG. 52 is a cross-sectional view of a portion of electrode 250 showing the arrangement of stylet hole 252, several electrical contacts, and several drug delivery holes (such as would be located at locations 255-259). The size of these holes can be adjusted to accommodate the pressure drop that would occur down stream from each hole, such that the desired amount of drug is released along the body of the CI electrode.

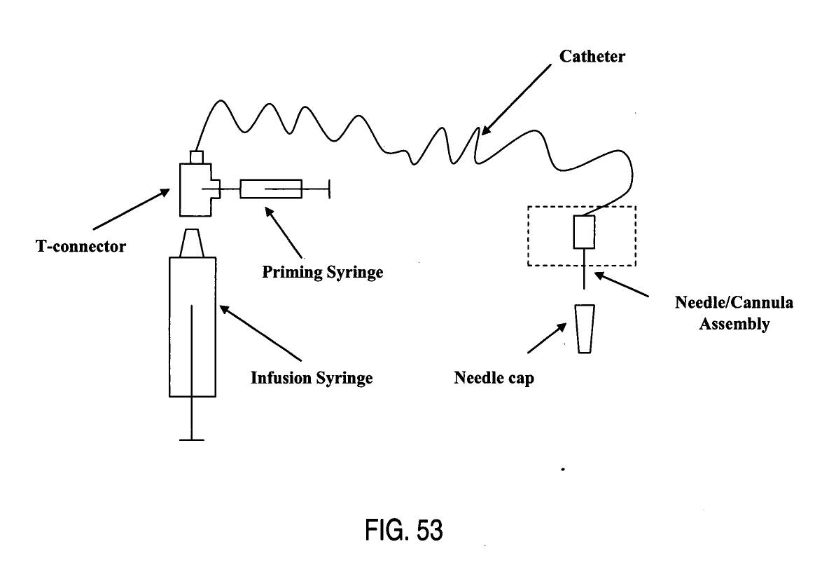

FIG. 53 is a schematic diagram of another apparatus, according to at least some embodiments, for delivering agents to the inner ear. In particular, the apparatus of FIG. 53 does not use a pump. The T-connector allows two kinds of fluid compositions to mix and be delivered to the patient simultaneously. The T-connector also acts as a port, and may have an attached (or internally incorporated) anti-bacterial filter.

FIG. 54 is a schematic diagram of an additional apparatus 10F, according to at least some embodiments, for delivering agents to the inner ear. Like components of apparatus 10F and previously described apparatuses have common reference numbers. Apparatus 10F of FIG. 54 includes a port 302 (having a septum 303) attached to a catheter 23. Needle 301 (attached to the pump 13 via another catheter 21 and an anti-bacterial filter with luer lock 16) and port 303 (attached to a catheter 23, which is attached to anti-bacterial filter 25, catheter 29 and needle 50) can be used instead of a quick disconnect.

In any of the embodiments discussed herein, the supply system and/or the fluid carrying system could be free of filters, quick disconnect fittings, or other components described herein. Similarly, the entire apparatus could be free of filters, including those discussed herein.

Numerous characteristics, advantages and embodiments of the invention have been described in detail in the foregoing description with reference to the accompanying drawings. However, the disclosure is illustrative only and the invention is not limited to the illustrated embodiments. Various changes and modifications may be effected therein by one skilled in the art without departing from the scope or spirit of the invention. Although example materials and dimensions have been provided, the invention is not limited to such materials or dimensions unless specifically required by the language of a claim. The elements and uses of the previously-described embodiments can be rearranged and combined in manners other than specifically described above, with any and all permutations within the scope of the invention. The methods and apparatuses described are not limited to use with an inner ear, or to use in a human. As used herein (including the claims), “in fluid communication” means that fluid can flow from one component to another; such flow may be by way of one or more intermediate (and not specifically mentioned) other components. As also used herein (including the claims), “coupled” includes two components that are attached (movably or fixedly) by one or more intermediate components.

1. An apparatus for sustained delivery of a drug or other agent to an inner ear or other tissue of a human or an animal, comprising:
a needle including a shaft with proximal and distal ends, the shaft including an internal duct in fluid communication with an opening formed in the distal end;
an insertion stop coupled to and extending outward from the needle shaft, a portion of the needle shaft proximate to and including the distal end extending free of the insertion stop; and
a catheter coupled to the needle shaft proximal end and including a lumen in fluid communication with the needle shaft internal duct, the lumen being formed from a fluoropolymer.

2. The apparatus of claim 1, further comprising a flange attached to the needle shaft at or near the proximal end thereof, said flange being at least partially encased within the catheter.

3. The apparatus of claim 2, wherein the flange is formed from a metal.

4. The apparatus of claim 2, wherein the flange is formed from a polymer.

5. The apparatus of claim 1, wherein the needle shaft distal end is of a size between about 22 gauge and about 35 gauge.

6. The apparatus of claim 1, wherein the needle shaft extends from an end of the catheter, and wherein the insertion stop is attached to the catheter around the catheter end.

7. The apparatus of claim 1, wherein the catheter includes at least one suture anchor.

8. The apparatus of claim 1, further comprising at least one in-line anti-bacterial filter in fluid communication with the catheter.

9. The apparatus of claim 8, wherein the at least one in-line filter includes a three-dimensional filter element.

10. The apparatus of claim 9, wherein the three-dimensional filter element is formed from a metal or a polymer.

11. The apparatus of claim 1, further comprising a quick-disconnect coupling having mating first and second portions, and wherein the catheter is attached to and in fluid communication with the first portion.

12. The apparatus of claim 11, wherein the first portion includes a septum and the second portion includes a needle positioned to pierce the septum when the first and second portions are mated, and wherein the first and second portions include internal fluid passageways formed from a fluoropolymer.

13. The apparatus of claim 1, further comprising a syringe housed within a micro-infusion pump, said syringe being in fluid communication with the catheter lumen.

14. The apparatus of claim 1, further comprising an osmotic pump in fluid communication with the catheter lumen.

15. The apparatus of claim 1, further comprising a port in fluid communication with the catheter lumen.

16. The apparatus of claim 1, wherein the catheter includes multiple lumens.

17. The apparatus of claim 1, wherein the needle shaft distal end is sharpened and configured to pierce a membrane or tissue of a human or an animal.

18. The apparatus of claim 1, wherein the needle shaft distal end is blunt and configured for insertion into a surgically prepared hole in a human or an animal bone.

19. The apparatus of claim 18, wherein the needle shaft is bent.

20. The apparatus of claim 18, wherein the insertion stop is formed from one or more porous biocompatible materials configured for fusion to the human or animal bone.

21. An apparatus for sustained delivery of a drug or other agent to an inner ear or other tissue of a human or an animal, comprising:
a needle including a shaft with proximal and distal ends, the shaft including an internal duct in fluid communication with an opening formed in the distal end;
an insertion stop coupled to and extending outward from the needle shaft, a portion of the needle shaft proximate to and including the distal end extending free of the insertion stop;
a catheter coupled to the needle shaft proximal end and including a lumen in fluid communication with the needle shaft internal duct;
at least one in-line anti-bacterial filter in fluid communication with the catheter;
a dispensing device configurable to automatically dispense the drug or other agent at rates of between 1 nanoliter/hour through 200 microliters/hour over a period of at least one hour.

22. The apparatus of claim 21, wherein the at least one in-line filter includes a three-dimensional filter element.

23. The apparatus of claim 22, wherein the three-dimensional filter element is formed from a metal or a polymer.

24. The apparatus of claim 23, wherein the at least one in-line filter includes first and second rigid tubular connectors, and wherein
the three-dimensional filter element is positioned between the first and second connectors,
at least one layer of a polymer material encases the three-dimensional filter element and portions of the first and second connectors adjacent to the three-dimensional filter element, and
the at least one polymer material layer and the first and second connectors form a fluid-tight conduit.

25. The apparatus of claim 24, wherein the needle shaft, the catheter lumen, the first and second connectors, the at least one polymer material and the three-dimensional filter element are formed from one or more materials selected from the group that includes titanium, stainless steel and fluoropolymers.

26. The apparatus of claim 24, wherein at least one of the tubular connectors has a flared end.

27. The apparatus of claim 24, wherein at least one of the tubular connectors has a barbed end.

28. The apparatus of claim 23, wherein the three-dimensional filter element is contained within a rigid housing formed from a metal or a polymer.

29. The apparatus of claim 21, further comprising a quick disconnect fitting in fluid communication with the catheter.

30. The apparatus of claim 29, wherein the quick disconnect fitting comprises internal fluid passages formed from a fluoropolymer.

31. The apparatus of claim 30, further comprising a second catheter and a luer connector, wherein
the second catheter is in fluid communication with the quick disconnect fitting,
the second catheter includes an internal lumen formed from a fluoropolymer, and
the luer connector includes internal fluid passages formed from a fluoropolymer.

32. The apparatus of claim 31, further comprising a syringe in fluid communication with the luer connector, the syringe including a barrel having an interior surface, wherein the interior barrel surface is formed from at least one of a fluoropolymer and acid-washed glass.

33. The apparatus of claim 21, wherein the needle shaft distal end is sharpened and configured to pierce a membrane or tissue of a human or an animal.

34. The apparatus of claim 21, wherein the needle shaft distal end is blunt and configured for insertion into a surgically prepared hole in a human or an animal bone.

35. The apparatus of claim 34, wherein the needle shaft is bent.

36. The apparatus of claim 34, wherein the insertion stop is formed from one or more porous biocompatible materials configured for fusion to the human or animal bone.

37. The apparatus of claim 21, further comprising a flange attached to the needle shaft at or near the proximal end thereof, said flange being at least partially encased within the catheter.

38. The apparatus of claim 21, wherein the needle shaft extends from an end of the catheter, and wherein the insertion stop is attached to the catheter around the catheter end.

39. An apparatus for sustained delivery of a drug or other agent to an inner ear or other tissue of a human or an animal, comprising:
a needle including a shaft with proximal and distal ends, the shaft including an internal duct in fluid communication with an opening formed in the distal end;
an insertion stop coupled to and extending outward from the needle shaft, a portion of the needle shaft proximate to and including the distal end extending free of the insertion stop;
a catheter coupled to the needle shaft proximal end and including a lumen in fluid communication with the needle shaft internal duct;
at least one in-line anti-bacterial filter in fluid communication with the catheter; and
a quick disconnect fitting in fluid communication with the catheter.

40. The apparatus of claim 39, wherein the needle shaft extends from an end of the catheter, and wherein the insertion stop is attached to the catheter around the catheter end.

41. The apparatus of claim 39, further comprising a flange attached to the needle shaft at or near the proximal end thereof, said flange being at least partially encased within the catheter.

42. The apparatus of claim 39, wherein the needle shaft, the catheter lumen, internal fluid passageways of the at least one in-line anti-bacterial filter and internal fluid passageways of the quick disconnect fitting are formed from one or more materials selected from the group that includes titanium, stainless steel and fluoropolymers.

43. The apparatus of claim 39, wherein the catheter includes at least one suture anchor.

44. The apparatus of claim 39, wherein the needle shaft distal end is sharpened and configured to pierce a membrane or tissue of a human or an animal.

45. The apparatus of claim 39, wherein the needle shaft distal end is blunt and configured for insertion into a surgically prepared hole in a human or an animal bone.

46. The apparatus of claim 45, wherein the needle shaft is bent.

47. The apparatus of claim 45, wherein the insertion stop is formed from one or more porous biocompatible materials configured for fusion to the human or animal bone.

48. An apparatus for sustained delivery of a drug or other agent to an inner ear or other tissue of a human or an animal, comprising:
a needle including a bent shaft with proximal and distal ends, the shaft including an internal duct in fluid communication with an opening formed in the distal end;
an insertion stop coupled to and extending outward from the needle shaft, a portion of the needle shaft proximate to and including the distal end extending free of the insertion stop; and
a catheter coupled to the needle shaft proximal end and including a lumen in fluid communication with the needle shaft internal duct.

49. The apparatus of claim 48, wherein the needle shaft is bent at an angle of approximately 100°.

50. The apparatus of claim 48, wherein the portion of the needle shaft extending free of the insertion stop is straight, and wherein a portion of the needle shaft having the bend is located on a proximal side of the insertion stop.

51. The apparatus of claim 50, wherein the needle shaft is bent at an angle of approximately 100°.

52. The apparatus of claim 48, wherein the distal end is pointed and configured for piercing a membrane or other tissue of a human or animal.

53. The apparatus of claim 48, wherein the needle has a size between 22 gauge and 35 gauge.

54. The apparatus of claim 48, wherein the distal end is located a distance of between 0.5 mm and 2.0 mm from the insertion stop.

55. An apparatus for sustained delivery of a drug or other agent to an inner ear or other tissue of a human or an animal, comprising:
a needle including a shaft with proximal and distal ends, the shaft including an internal duct in fluid communication with an opening formed in the distal end;
a flexible insertion stop coupled to and extending outward from the needle shaft, a portion of the needle shaft proximate to and including the distal end extending free of the insertion stop; and
a catheter coupled to the needle shaft proximal end and including a lumen in fluid communication with the needle shaft internal duct.

56. The apparatus of claim 55, wherein the insertion stop is transparent.

57. The apparatus of claim 55, wherein the insertion stop is formed from a silicone elastomer.

58. The apparatus of claim 55, wherein the needle shaft is bent.

59. The apparatus of claim 55, wherein the insertion stop has a thickness of between about 0.2 mm and about 1 mm.

60. The apparatus of claim 55, wherein the insertion stop is round and has a diameter of about 1 mm to about 3 mm.

61. The apparatus of claim 55, wherein the needle shaft is bent and the insertion stop is transparent.

62. The apparatus of claim 61, wherein the insertion stop has a thickness of between about 0.2 mm and about 1 mm.

63. The apparatus of claim 62, wherein the insertion stop is round and has a diameter of about 1 mm to about 3 mm.

64. The apparatus of claim 63, wherein the needle has a size between 22 gauge and 35 gauge.

65. The apparatus of claim 64, wherein the distal end is located a distance of between 0.5 mm and 2.0 mm from the insertion stop.

66. The apparatus of claim 65, wherein the needle shaft is bent at an angle of approximately 100°.

67. A port configured for sub-cutaneous or partially subcutaneous implantation in a human or an animal, comprising:
a reservoir having a cavity formed therein;
a cap, the cap and reservoir being cooperable to attach to one another;
an elastomeric septum covering the cavity when secured to the reservoir by the cap; and
at least one attachment fixture extending from the port, the at least one attachment fixture including an opening configured to receive a bone screw, the opening sufficiently spaced from the port to avoid cracking bone adjacent a depression in said bone in which the reservoir rests when the port is screwed in place.

68. The port of claim 67, further comprising an outlet tube having an internal passageway in fluid communication with the reservoir cavity.

69. The port of claim 67, wherein the reservoir includes one or more fixtures of a first type and the cap includes one or more fixtures of a second type, and wherein the first and second type fixtures cooperate to attach the cap to the reservoir.

70. The port of claim 69, wherein the first type fixtures include one or more tabs and the second type fixtures include one or more slots.

71. The port of claim 67, wherein the at least one attachment fixture is attached to the reservoir.

72. The port of claim 67, wherein the port comprises a three-dimensional antibacterial filter.

73. The port of claim 72, wherein the filter is inside the reservoir.

74. The port of claim 72, wherein the filter is outside the reservoir.

75. The port of claim 72, further comprising an outlet tube having an internal passageway in fluid communication with the reservoir cavity, wherein the outlet tube is external to the reservoir, and wherein the filter is inside the outlet tube.

76. The port of claim 67, wherein the reservoir cavity is tapered.

77. The port of claim 67, further comprising:
a catheter having a lumen in fluid communication with the reservoir cavity; and
a cochlear implant electrode having an internal duct in fluid communication with the catheter lumen and a plurality of outlet holes, wherein the outlet holes are in fluid communication with the duct.

78. The port of claim 67, further comprising:
a catheter having a lumen in fluid communication with the reservoir cavity; and
a needle having an internal duct and an insertion stop, wherein the internal duct is in fluid communication with the catheter lumen.

79. A port configured for sub-cutaneous or partially subcutaneous implantation in a human or an animal, comprising:
a reservoir having a cavity formed therein;
an elastomeric septum covering the cavity;
a catheter having a lumen in fluid communication with the reservoir cavity; and
a cochlear implant electrode having an internal duct in fluid communication with the catheter lumen and a plurality of outlet holes, wherein the outlet holes are in fluid communication with the duct.

80. The port of claim 79, wherein the port comprises a three-dimensional antibacterial filter.

81. The port of claim 80, wherein the filter is inside the reservoir.

82. The port of claim 80, wherein the filter is outside the reservoir.

83. The port of claim 80, further comprising an outlet tube having an internal passageway in fluid communication with the reservoir cavity, wherein the outlet tube is external to the reservoir, and wherein the filter is inside the outlet tube.

84. The port of claim 79, wherein the reservoir cavity is tapered.

85. A port configured for sub-cutaneous or partially subcutaneous implantation in a human or an animal, comprising:
a reservoir having a cavity formed therein;
an elastomeric septum covering the cavity;
a catheter having a lumen in fluid communication with the reservoir cavity;
a needle including a shaft with proximal and distal ends, the needle shaft including an internal duct in fluid communication with an opening formed in the distal end and with the catheter lumen; and wherein
the needle shaft bent,
the needle shaft includes an insertion stop coupled to and extending outward from the needle shaft, a portion of the needle shaft proximate to and including the distal end extending free of the insertion stop, or
the needle shaft is bent and includes an insertion stop coupled to and extending outward from the needle shaft, a portion of the needle shaft proximate to and including the distal end extending free of the insertion stop.

86. The port of claim 85, wherein the port comprises a three-dimensional antibacterial filter.

87. The port of claim 86, wherein the filter is inside the reservoir.

88. The port of claim 86, wherein the filter is outside the reservoir.

89. The port of claim 86, further comprising an outlet tube having an internal passageway in fluid communication with the reservoir cavity, wherein the outlet tube is external to the reservoir, and wherein the filter is inside the outlet tube.

90. The port of claim 85, wherein the reservoir cavity is tapered.

91. A port configured for sub-cutaneous or partially subcutaneous implantation in a human or an animal, comprising:
a reservoir having a cavity formed therein;
an elastomeric septum covering the cavity; and
a three-dimensional antibacterial filter inside the reservoir.

92. The port of claim 91, wherein the reservoir cavity is tapered.

93. A port configured for sub-cutaneous or partially subcutaneous implantation in a human or an animal, comprising:
a reservoir having a cavity formed therein;
an elastomeric septum covering the cavity when secured to the reservoir by the cap; and
an outer surface formed from a porous biocompatible material coated with biomaterials that allow binding of human or animal cells to the outer surface.

94. The port of claim 93, wherein the biomaterials include at least one of extracellular matrices such as collagen, laminin, glycosoaminoglycan, fibronectin and fibronectin fragments such as peptides that contain the ArgGlyAsp epitope for cell adhesion.

95. The port of claim 93, wherein the port comprises a three-dimensional antibacterial filter.

96. The port of claim 95, wherein the filter is inside the reservoir.

97. The port of claim 95, wherein the filter is outside the reservoir.

98. The port of claim 95, further comprising an outlet tube having an internal passageway in fluid communication with the reservoir cavity, wherein the outlet tube is external to the reservoir, and wherein the filter is inside the outlet tube.

99. The port of claim 93, wherein the reservoir cavity is tapered.

100. A method of treating an inner ear of a human or an animal, the method comprising the steps of:
inserting an injection end of a needle into the inner ear, the needle being in fluid communication with a catheter, and the catheter being in fluid communication with a source of an agent, wherein the agent is selected from the group consisting of an NMDA receptor antagonist, a subtype-specific NMDA receptor antagonist; an anxiolytic, an anti-depressant, a selective serotonin reabsorption inhibitor, an anti-convulsant, an anti-epilepsy drug, an anti-seizure drug, a calcium channel blocker, a sodium channel blocker, an anti-migraine agent, a muscle relaxant, a hypnotic, an anti-inflammatory steroid, and mixtures thereof; and
dispensing the agent through the needle over a period of at least one hour and without removing the injection end from the inner ear.

101. The method of claim 100, wherein the agent includes one or more of carbamathione, an N-methyl analog of carbamathione or an N-benzyl analog of carbamathione.

102. The method of claim 100, wherein the dispensing step includes dispensing the agent as a bolus injection or as multiple doses given as an intermittent or continuous infusion.

103. The method of claim 100, wherein the agent is selected from the group consisting of alprazolam, memantine, cyclandelate, caroverine, lidocaine, tocainide, gabapentin, mephobarbital, sodium pentobarbital, lorazepam, clonazepam, clorazepate dipotassium, diazepam, tiagabine, β-hydroxypropionic acid, phenyltoin, fosphenyloin sodium, lamotrigine, methsuximide, ethosuximide, carbamazepine, divalproex sodium, felbamate, levetiracetam, primidone, zonisamide, topiramate, sodium valproate, LY 274614, LY 235959, LY 233053, NPC 12626, reduced or oxidized glutathione, carbamathione, the N-methyl or N-benzyl analogs of carbamathione, AP5, CPP, CGS-19755, CGP-37849, CGP-39551, SDZ 220-581, S-nitrosoglutathione, amantadine, aptiganel, caroverine, dextrophan, dextromethorphan, fullerenes, gacyclidine (GK-11), ibogaine, ketamine, dizocilpine (MK-801), neramexane (MRZ 2/579), NPS 1506 (delucemine), phencyclidine, tiletamine, remacemide, acamprosate, arcaine, conantokin-G, eliprodil (SL 82-0715), haloperidol, ifenprodil, traxoprodil (CP-101,606), Ro 25-6981, aminocyclopropanecarboxylic acid (ACPC), 7-chlorokynurenic acid, D-cycloserine, gavestinel (GV-150526), GV-196771A, licostinel (ACEA 1021), MRZ-2/576, L-701,324, HA-966, ZD-9379, sodium nitroprusside, ebselen, disulfiram, CNQX, DNQX, argiotoxin636, Co 101244 (PD 174494, Ro 63-1908), despiramine, philanthotoxin343, Ro 04-5595, spermine, spermidine, NVP-AAM077, nortriptyline, fluoxetine, paroxetine, trimipramine, oxacarbazepine, eperisone, misoprostol, pregnenolone, triamcinolone, or methylprednisolone and mixtures thereof.

104. The method of claim 100, wherein the agent includes one or more of gacyclidine, traxoprodil, ifenprodil and eliprodil.

105. The method of claim 100, wherein the step of inserting includes inserting the injection end of the needle into the inner ear of a patient suffering an inner ear disorder selected from the group of disorders selected from the group consisting of tinnitus, vertigo, noise-induced hearing loss, drug-induced hearing loss, chronic ear pain, neurodegeneration, Meniere's disease, surgical trauma or neurodegeneration of the auditory nerve, sprial ganglion or neurological connections therein.

106. The method of claim 100, wherein the agent is a neuroprotectant or an anti-apoptic agent.

107. The method of claim 100, wherein the agent includes one or more agents capable of restoring hearing or preventing progression of an ongoing chronic hearing disorder.

108. The method of claim 100, wherein the step of inserting includes inserting the injection end of the needle into the inner ear of a patient after noise-induced or other type of trauma, and wherein the agent is a neuroprotectant or an anti-apoptic agent.

109. The method of claim 100, wherein the step of inserting includes inserting the injection end of the needle into the inner ear of a patient after drug or chemically induced trauma, and wherein the agent is a neuroprotectant or an anti-apoptic agent.

110. A method of treating a disorder comprising the step of:
injecting a therapeutic agent directly into an area of a patient's body to be treated with the agent using a device that includes
a needle including a shaft with proximal and distal ends, the shaft including an internal duct in fluid communication with an opening formed in the distal end, and
a catheter coupled to the needle shaft proximal end and including a lumen in fluid communication with the needle shaft internal duct, the lumen being formed from a fluoropolymer.

111. The method of claim 110, wherein the therapeutic agent is selected from the group consisting of an NMDA receptor antagonist, a subtype-specific NMDA receptor antagonist; an anxiolytic, an anti-depressant, a selective serotonin reabsorption inhibitor, an anti-convulsant, an anti-epilepsy drug, an anti-seizure drug, a calcium channel blocker, a sodium channel blocker, an anti-migraine agent, a muscle relaxant, a hypnotic, an anti-inflammatory steroid, and mixtures thereof.

112. The method of claim 110, wherein the agent is selected from the group consisting of alprazolam, memantine, cyclandelate, caroverine, lidocaine, tocainide, gabapentin, mephobarbital, sodium pentobarbital, lorazepam, clonazepam, clorazepate dipotassium, diazepam, tiagabine, β-hydroxypropionic acid, phenyltoin, fosphenyloin sodium, lamotrigine, methsuximide, ethosuximide, carbamazepine, divalproex sodium, felbamate, levetiracetam, primidone, zonisamide, topiramate, sodium valproate, LY 274614, LY 235959, LY 233053, NPC 12626, reduced or oxidized glutathione, carbamathione, the N-methyl or N-benzyl analogs of carbamathione, AP5, CPP, CGS-19755, CGP-37849, CGP-39551, SDZ 220-581, S-nitrosoglutathione, amantadine, aptiganel, caroverine, dextrophan, dextromethorphan, fullerenes, gacyclidine (GK-11), ibogaine, ketamine, dizocilpine (MK-801), neramexane (MRZ 2/579), NPS 1506 (delucemine), phencyclidine, tiletamine, remacemide, acamprosate, arcaine, conantokin-G, eliprodil (SL 82-0715), haloperidol, ifenprodil, traxoprodil (CP-101,606), Ro 25-6981, aminocyclopropanecarboxylic acid (ACPC), 7-chlorokynurenic acid, D-cycloserine, gavestinel (GV-150526), GV-196771A, licostinel (ACEA 1021), MRZ-2/576, L-701,324, HA-966, ZD-9379, sodium nitroprusside, ebselen, disulfiram, CNQX, DNQX, argiotoxin636, Co 101244 (PD 174494, Ro 63-1908), despiramine, philanthotoxin343, Ro 04-5595, spermine, spermidine, NVP-AAM077, nortriptyline, fluoxetine, paroxetine, trimipramine, oxacarbazepine, eperisone, misoprostol, pregnenolone, triamcinolone, or methylprednisolone and mixtures thereof.

113. A method of treating a disorder comprising the step of:
injecting a therapeutic agent directly into an area of a patient's body to be treated with the agent using a device that includes
a needle including a shaft with proximal and distal ends, the shaft including an internal duct in fluid communication with an opening formed in the distal end,
a catheter coupled to the needle shaft proximal end and including a lumen in fluid communication with the needle shaft internal duct;
at least one in-line anti-bacterial filter in fluid communication with the catheter;
a dispensing device configurable to automatically dispense the drug or other agent at rates of between a 1 nanoliter/hour through 200 microliters/hour over a period of at least one hour.

114. The method of claim 113, wherein the therapeutic agent is selected from the group consisting of an NMDA receptor antagonist, a subtype-specific NMDA receptor antagonist; an anxiolytic, an anti-depressant, a selective serotonin reabsorption inhibitor, an anti-convulsant, an anti-epilepsy drug, an anti-seizure drug, a calcium channel blocker, a sodium channel blocker, an anti-migraine agent, a muscle relaxant, a hypnotic, an anti-inflammatory steroid, and mixtures thereof.

115. The method of claim 113, wherein the agent is selected from the group consisting of alprazolam, memantine, cyclandelate, caroverine, lidocaine, tocainide, gabapentin, mephobarbital, sodium pentobarbital, lorazepam, clonazepam, clorazepate dipotassium, diazepam, tiagabine, β-hydroxypropionic acid, phenyltoin, fosphenyloin sodium, lamotrigine, methsuximide, ethosuximide, carbamazepine, divalproex sodium, felbamate, levetiracetam, primidone, zonisamide, topiramate, sodium valproate, LY 274614, LY 235959, LY 233053, NPC 12626, reduced or oxidized glutathione, carbamathione, the N-methyl or N-benzyl analogs of carbamathione, AP5, CPP, CGS-19755, CGP-37849, CGP-39551, SDZ 220-581, S-nitrosoglutathione, amantadine, aptiganel, caroverine, dextrophan, dextromethorphan, fullerenes, gacyclidine (GK-11), ibogaine, ketamine, dizocilpine (MK-801), neramexane (MRZ 2/579), NPS 1506 (delucemine), phencyclidine, tiletamine, remacemide, acamprosate, arcaine, conantokin-G, eliprodil (SL 82-0715), haloperidol, ifenprodil, traxoprodil (CP-101,606), Ro 25-6981, aminocyclopropanecarboxylic acid (ACPC), 7-chlorokynurenic acid, D-cycloserine, gavestinel (GV-150526), GV-196771A, licostinel (ACEA 1021), MRZ-2/576, L-701,324, HA-966, ZD-9379, sodium nitroprusside, ebselen, disulfiram, CNQX, DNQX, argiotoxin636, Co 101244 (PD 174494, Ro 63-1908), despiramine, philanthotoxin343, Ro 04-5595, spermine, spermidine, NVP-AAM077, nortriptyline, fluoxetine, paroxetine, trimipramine, oxacarbazepine, eperisone, misoprostol, pregnenolone, triamcinolone, or methylprednisolone and mixtures thereof.
